(19) United States
(12) Patent Application Publication
(10) Pub. No.: US 2008/0280883 A1
(43) Pub. Date: Nov. 13, 2008

(54) Muscarinic Receptor Antagonists

(76) Inventors: Mohammad Salman, Princeton, NJ (US); Pakala Kumara Savithru Sarma, Haryana (IN); Sankaranarayanan Dharmarajan, Haryana (IN); Anita Chugh, Delhi (IN); Suman Gupta, Haryana (IN)

Correspondence Address: RANBAXY INC., 600 COLLEGE ROAD EAST, SUITE 2100, PRINCETON, NJ, 08540, US

(21) Appl. No.: 11/575,825

(22) PCT Filed: Sep. 23, 2005
(86) PCT No.: PCT/IB2005/002823

§ 371 Date: Dec. 13, 2007

Related U.S. Application Data
(60) Provisional application No. 60/613,001, filed on Sep. 24, 2004.

(51) Int. Cl. A61K 31/551; A61K 31/439; C07D 221/22; C07D 265/30; A61P 11/00; A61K 31/5375; C07D 405/08

(52) U.S. Cl. 514/218; 546/112; 514/299; 544/175; 514/238.8; 540/575

(57) ABSTRACT

This present invention generally relates to muscarinic receptor antagonists which are useful, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### FIELD OF THE INVENTION

This present invention generally relates to muscarinic receptor antagonists, which are suitable, among other uses, for the treatment of various diseases of the respiratory, urinary and gastrointestinal systems mediated through muscarinic receptors. The invention also relates to the process for the preparation of disclosed compounds, pharmaceutical compositions containing the disclosed compounds, and the methods for treating diseases mediated through muscarinic receptors.

### BACKGROUND OF THE INVENTION

Muscarinic receptors as members of the G Protein Coupled Receptors (GPCRs) are composed of a family of 5 receptor sub-types (M₁, M₂, M₃, M₄ and M₅) and are activated by the neurotransmitter acetylcholine. These receptors are widely distributed on multiple organs and tissues and are critical to the maintenance of central and peripheral cholinergic neurotransmission. The regional distribution of these receptor sub-types in the brain and other organs has been documented (for example, the M₁ subtype is located primarily in neuronal tissues such as cereberal cortex and autonomic ganglia, the M₂ subtype is present mainly in the heart where it mediates cholinergically induced bradycardia, and the M₃ subtype is located predominantly on smooth muscle and salivary glands (Nature, 323, p. 411(1986); Science, 237, p. 527(1987)).

A review in Current Opinions in Chemical Biology, 3, p. 426(1999), as well as in Trends in Pharmacological Sciences, 22, p. 409(2001) by Eglen et. al., describes the biological potentials of modulating muscarinic receptor subtypes by ligands in different disease conditions, such as Alzheimer's Disease, pain, urinary disease condition, chronic obstructive pulmonary disease, and the like.

A review in J. Med. Chem., 43, p. 4333(2000), by Felder et. al. describes therapeutic opportunities for muscarinic receptors in the central nervous system and elaborates on muscarinic receptor structure and function, pharmacology and their therapeutic uses.

The pharmacological and medical aspects of the muscarinic class of acetylcholine agonists and antagonists are presented in a review in Molecules, 6, p. 142(2001).

Birdsall et al. in Trends in Pharmacological Sciences, 22, p. 215(2001) have also summarized the recent developments on the role of different muscarinic receptor subtypes using different muscarinic receptor of knock out mice.

Muscarinic agonists such as muscarine and pilocarpine and antagonists such as atropine have been known for over a century, but little progress has been made in the discovery of receptor subtype-selective compounds, making it difficult to assign specific functions to the individual receptors. Although classical muscarinic antagonists such as atropine are potent bronchodilators, their clinical utility is limited due to high incidence of both peripheral and central adverse effects such as tachycardia, blurred vision, dryness of mouth, constipation, dementia, etc. Subsequent development of the quarterly derivatives of atropine such as ipratropium bromide are better tolerated than parenterally administered options, but most of these are not ideal anti-cholinergic bronchodilators, due to lack of selectivity for muscarinic receptor sub-types, resulting in dose-limiting side-effects such as thirst, nausea, mydriasis and those associated with the heart such as tachycardia mediated by the M₂ receptor.

Annual Review of Pharmacological Toxicol., 41, p. 691(2001), describes the pharmacology of the lower urinary tract infections. Although anti-muscarinic agents such as oxybutynin and tolterodine that act non-selectively on muscarinic receptors have been used for many years to treat bladder hyperactivity, the clinical effectiveness of these agents has been limited due to the side effects such as dry mouth, blurred vision and constipation. Tolterodine is considered to be generally better tolerated than oxybutynin. (Steers et. al., in Curr. Opin. Invest. Drugs, 2, 268; Chapple et. al., in Urology, 55, 33; Steers et al., Adult and Pediatric Urology, ed. Gillenwatteret al., pp 1220-1325, St. Louis, Mo.; Mosby. 3^{rd} edition (1996)).

There remains a need for development of new highly selective muscarinic antagonists which can interact with distinct subtypes, thus avoiding the occurrence of adverse effects.

WO 04/005252 discloses azabicyclo derivatives described as muscarinic receptor antagonists. WO 04/004629, WO 04/052857, WO 04/067510, WO 04/014853, WO 04/014363 discloses 3,6-disubstituted azabicyclo [3.1.0] hexane derivatives described as useful muscarinic receptor antagonists. WO 04/056811 discloses flaxavate derivatives as muscarinic receptor antagonists. WO 04/056810 discloses xanthene derivatives as muscarinic receptor antagonists. WO 04/056767 discloses 1-substituted-3-pyrrolidine derivatives as muscarinic receptor antagonists. WO 04/089363, WO 04/089898, WO 04/069835, WO 04/089900 and WO 04/089364 discloses substituted azabicyclohexane derivatives as muscarinic receptor antagonists.

Compounds having antagonistic activity against muscarinic receptors have been described in Japanese patent application Laid Open Number 92921/1994 and 135958/1994; WO 93/16048; U.S. Pat. No. 3,176,019; GB 940,540; EP 0325 571; WO 98/29402; EP 0801067; EP 0388054; WO 9109013; U.S. Pat. No. 5,281,601. Also, U.S. Pat. Nos. 6,174,900, 6,130,232 and 5,948,792; WO 97/45414 are related to 1,4-disubstituted piperidine derivatives; WO 98/05641 describes fluorinated, 1,4-disubstitued piperidine derivatives; WO 93/16018 and WO96/33973 are other references of interest. U.S. Pat. No. 5,397,800 discloses 1-azabicyclo[2.2.1]heptanes. U.S. Pat. No. 5,001,160 describes 1-aryl-1-hydroxy-1-substituted-3-(4-substituted-1-piperazinyl)-2-propanones. WO 01/42213 describes 2-biphenyl-4-piperidinyl ureas. WO 01/42212 describes carbamate derivatives. WO 01/90081 describes amino alkyl lactam. WO 02/53564 describes novel quinuclidine derivatives. WO 02/00652 describes carbamates derived from arylalkyl amines. WO 02/06241 describes 1,2,3,5-tetrahydrobenzo(c)azepin-4-one derivatives. U.S. application No. 20030105071 describes thiazole and other heterocyclic ligands for mammalian dopamine, muscarinic and serotonic receptors and transporters, and method of use thereof.

J. Med. Chem., 44, p. 984 (2002), describes cyclohexylmethylpiperidinyl-triphenylpropioamide derivatives as selective M₃ antagonist discriminating against the other receptor subtypes. J. Med. Chem., 36, p. 610 (1993), describes the synthesis and antimuscarinic activity of some 1-cycloalkyl-1-hydroxy-1-phenyl-3-(4-substituted piperazinyl)-2-propanones and related compounds. J. Med. Chem., 34, p. 3065 (1991), describes analogues of oxybutynin, synthesis and antimuscarinic activity of some substituted 7-amino-1-hydroxy-5-heptyn-2-ones and related compounds.

### SUMMARY OF THE INVENTION

In one aspect, there are provided muscarinic receptor antagonists, which can be useful as safe and effective therapeutic or prophylactic agents for the treatment of various diseases of the respiratory, urinary and gastrointestinal systems. Also provided are processes for synthesizing such compounds.

In another aspect, pharmaceutical compositions containing such compounds are provided together with acceptable carriers, excipients or diluents which can be useful for the treatment of various diseases of the respiratory, urinary and gastrointestinal systems.

The enantiomers, diastereomers, N-oxides, polymorphs, pharmaceutically acceptable salts and pharmaceutically acceptable solvates of these compounds as well as metabolites having the same type of activity are also provided, as well as pharmaceutical compositions comprising the compounds, their metabolites, enantiomers, diastereomers, N-oxides, polymorphs, solvates or pharmaceutically acceptable salts thereof, in combination with a pharmaceutically acceptable carrier and optionally included excipients.

Other aspects will be set forth in the description which follows, and in part will be apparent from the description or may be learnt by the practice of the invention.

In accordance with one aspect, there are provided compounds having the structure of Formula I:

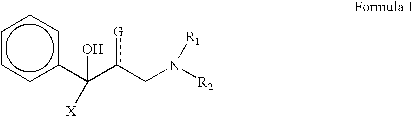

and their pharmaceutically acceptable salts, pharmaceutically acceptable solvates, esters, enantiomers, diastereomers, N-oxides, polymorphs, metabolites, wherein [symbol] represents an optional double bond;
X can be alkyl, alkenyl, alkynyl, cycloalkyl, aryl, heteroaryl, heterocyclyl, heterocyclylalkyl, or heteroarylalkyl.
R₁ can be hydrogen, alkyl, alkenyl, alkynyl, cycloalkyl, alkoxy, aryloxy; —(CH₂)₀₋₂-heterocyclylalkyl, or —(CH₂)₀₋₂-heteroarylalkyl.
R₂ can be —(CH₂)₀₋₂-heteroaryl, —(CH₂)₀₋₂-heterocyclyl, or —(CH₂)₀₋₂-aryl, or R₁ and R₂ may together combine to form a (un)saturated monocyclic or bicyclic ring system containing 0-4 heteroatoms (O, N or S) wherein the ring can be optionally substituted with one or more of alkyl; alkenyl; alkynyl; cycloalkyl; alkaryl; alkoxy; aryloxy; halogen (F, Cl, Br, I); heterocyclyl; heteroaryl; heterocyclylalkyl; heteroarylalkyl; carboxy; nitro; cyano; —C(═O)NRₓR_{y} (wherein Rₓ and R_{y} can be hydrogen, alkyl, alkenyl, alkynyl, cycloalkyl, hydroxy, alkoxy, aryloxy, —(CH₂)₀₋₂-heteroaryl, or —(CH₂)₀₋₂-heterocyclyl); —NRₓR_{y} (wherein Rₓ and R_{y} are as defined above), or —O—C(═O)NRₓR_{y} (wherein Rₓ and R_{y} are as defined above).

G can be —OR (wherein R represents hydrogen or unsubstituted lower (C₁-C₆) alkyl); —NOR (wherein R is the same as defined above); —NHYR′ (wherein R′ is hydrogen, alkyl or aryl and Y is —C(═O), —SO or —SO₂); or oxygen (provided that R₁ and R₂ together does not form a pyrrolidine, 4-hydroxy piperidine, 4-pyrrolidinyl piperidine, piperazine or azabicyclo[3.1.0]hexane ring).

The following definitions apply to terms as used herein.

The term “alkyl,” unless otherwise specified, refers to a monoradical branched or unbranched saturated hydrocarbon chain having from 1 to 20 carbon atoms. This term is exemplified by groups such as methyl, ethyl, n-propyl, iso-propyl, n-butyl, iso-butyl, t-butyl, n-hexyl, n-decyl, tetradecyl, and the like. Alkyl may further be substituted with one or more substituents such as alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryloxy, aminosulfonyl, aminocarbonylamino, —NRₓR_{y}, —C(═O)NRₓR_{y}, —O—C(═O)NRₓR_{y} (wherein Rₓ and R_{y} are the same as defined earlier), nitro, —S(O)ₙR₃ wherein R₃ is alkyl, aryl or heteroaryl. Unless otherwise constrained by the definition, all substituents may be further substituted by 1-3 substituents chosen from alkyl, carboxy, —NRₓR_{y}, —C(═O)NRₓR_{y}, —O—C(═O)NRₓR_{y} (wherein Rₓ and R_{y} are the same as defined earlier), hydroxy, alkoxy, halogen, CF₃, cyano, and —S(O)ₙR₃, where R₃ is the same as defined earlier and n is 0, 1 or 2. Alkyl groups may also be interrupted by 1-5 atoms of groups independently chosen from oxygen, sulfur and —NRₐ—, where Rₐ is chosen from hydrogen, alkyl, cycloalkyl, alkenyl, cycloalkenyl, alkynyl, aryl. Unless otherwise constrained by the definition, all substituents may be further substituted by 1-3 substituents chosen from alkyl, carboxy, —NRₓR_{y}, —C(═O)NRₓR_{y}, —O—C(═O)NRₓR_{y} (wherein Rₓ and R_{y} are the same as defined earlier), hydroxy, alkoxy, halogen, CF₃, cyano, and —S(O)ₙR₃ where n and R₃ are the same as defined earlier.

The term “alkenyl,” unless otherwise specified, refers to a monoradical of a branched or unbranched unsaturated hydrocarbon group preferably having from 2 to 20 carbon atoms with cis or trans geometry. Particular alkenyl groups include ethenyl or vinyl, 1-propylene or allyl, iso-propylene, bicyclo[2.2.1]heptene, and the like. In the event that alkenyl is attached to the heteroatom, the double bond cannot be alpha to the heteroatom. Alkenyl may further be substituted with one or more substituents such as alkyl, alkynyl, alkoxy, cycloalkyl, cycloalkenyl, acyl, acylamino, acyloxy, —NRₓR_{y}, —C(═O)NRₓR_{y}, —O—C(═O)NRₓR_{y} (wherein Rₓ and R_{y} are the same as defined earlier), alkoxycarbonylamino, azido, cyano, halogen, hydroxy, oxo, thiocarbonyl, carboxy, arylthio, thiol, alkylthio, aryl, alkaryl, aryloxy, heterocyclyl, heteroaryl, heterocyclyl alkyl, heteroaryl alkyl, aminosulfonyl, aminocarbonylamino, alkoxyamino, nitro, S(O)ₙR₃ (wherein R₃ is the same as defined earlier). Unless otherwise constrained by the definition, all substituents may optionally be further substituted by 1-3 substituents chosen from alkyl, carboxy, hydroxy, alkoxy, halogen, —CF₃, cyano, —NRₓR_{y}, —C(═O)NRₓR_{y}, —O—C(═O)NRₓR_{y} (wherein Rₓ and R_{y} are the same as defined earlier) and —S(O)ₙR₃, where R₃ and n are the same as defined earlier.

The term “alkynyl,” unless otherwise specified, refers to a monoradical of an unsaturated hydrocarbon, preferably having from 2 to 20 carbon atoms. Particular alkynyl groups include, for example, ethynyl, propargyl or propynyl, and the like. In the event that alkynyl is attached to the heteroatom, the triple bond cannot be alpha to the heteroatom. Alkynyl may further be substituted with one or more substituents selected from the group consisting of alkyl, alkenyl, alkoxy, cycloalkyl, cycloalkenyl, acyl, acylamino, acyloxy, alkoxycarbonylamino, azido, cyano, halogen, hydroxy, oxo, thiocarbonyl, carboxy, arylthio, thiol, alkylthio, aryl, alkaryl, aryloxy, aminosulfonyl, aminocarbonylamino, nitro, heterocyclyl, heteroaryl, heterocyclylalkyl, heteroarylalkyl, —NRₓR_{y}, —C(═O)NRₓR_{y}, —OC(═O)NRₓR_{y} (wherein Rₓ and R_{y} are the same as defined earlier), —S(O)ₙR₃ wherein R₃ is the same as defined earlier. Unless otherwise constrained by the definition, all substituents may optionally be further substituted by 1-3 substituents chosen from alkyl, carboxy, carboxyalkyl, hydroxy, alkoxy, halogen, CF₃, —NRₓR_{y}, —C(═O)NRₓR_{y}, —O—C(═O)NRₓR_{y} (wherein Rₓ and R_{y} are the same as defined earlier), cyano, and —S(O)ₙR₃, where R₃ and n are the same as defined earlier.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and bicyclo [2.2.1]heptane, or cyclic alkyl groups to which is fused an aryl group, (for example, indane, and the like. Spiro groups are also envisioned. Cycloalkyl groups may further be substituted with one or more substituents such as alkyl, alkenyl, alkynyl, alkoxy, cycloalkyl, cycloalkenyl, acyl, acylamino, acyloxy, alkoxycarbonylamino, azido, cyano, halogen, hydroxy, oxo, thiocarbonyl, carboxy, carboxyalkyl, arylthio, thiol, alkylthio, aryl, alkaryl, aryloxy, aminosulfonyl, aminocarbonylamino, —NRₓR_{y}, —C(═O)NRₓR_{y}, —O—C(═O)NRₓR_{y} (wherein Rₓ and R_{y} are the same as defined earlier), nitro, heterocyclyl, heteroaryl, heterocyclylalkyl, heteroarylalkyl, S(O)ₙR₃ wherein R₃ is same as defined earlier. Unless otherwise constrained by the definition, all substituents may optionally be further substituted by 1-3 substituents chosen from alkyl, carboxy, hydroxy, alkoxy, halogen, CF₃, —NRₓR_{y}, —C(═O)NRₓR_{y}, O—C(═O)NRₓR_{y} (wherein Rₓ and R_{y} are the same as defined earlier), cyano and —S(O)ₙR₃, where R₃ and n are the same as defined earlier.

The term “alkoxy” denotes the group O-alkyl wherein alkyl is the same as defined above. The term “alkaryl” refers to aryl linked through alkyl (wherein alkyl is the same as defined above) portion and the said alkyl portion contains carbon atoms from 1-6 and aryl is as defined below.

The term “aryl” herein refers to a carbocyclic aromatic group, (for example, phenyl, biphenyl or naphthyl ring and the like optionally substituted with 1 to 3 substituents selected from the group consisting of halogen (F, Cl, Br, I), hydroxy, alkyl, alkenyl, alkynyl, cycloalkyl, alkoxy, acyl, aryloxy, cyano, nitro, —NRₓR_{y}, —C(═O)NRₓR_{y}, —O—C(═C))NRₓR_{y} (wherein Rₓ and R_{y} are the same as defined earlier), carboxy, heterocyclyl, heteroaryl, heterocyclylalkyl, heteroarylalkyl or amino carbonylamino. The aryl group may optionally be fused with heterocyclyl, cycloalkyl or heteroaryl ring system.

The term “carboxy” as defined herein refers to —C(═O)O—R₄ wherein R₄ is selected from the group consisting of hydrogen, alkyl, alkenyl, alkynyl and cycloalkyl.

The term “heteroaryl,” unless otherwise specified, refers to an aromatic ring structure containing 5 to 7 ring atoms, or a bicyclic aromatic group having 8 to 12 ring atoms, with one or more heteroatom(s) (N, O or S) optionally substituted with 1 to 3 substituent(s) such as halogen (F, Cl, Br, I), hydroxy, alkyl, alkenyl, alkynyl, cycloalkyl, acyl, carboxy, aryl, alkoxy, alkaryl, cyano, nitro, aminocarbonylamino, —NRₓR_{y}, —C(═O)NRₓR_{y} and —O—C(═O)NRₓR_{y} (wherein Rₓ and R_{y} are the same as defined earlier). Examples of heteroaryl groups include pyridinyl, pyridazinyl, pyrimidinyl, pyrrolyl, oxazolyl, thiazolyl, thienyl, isoxazolyl, triazinyl, furanyl, benzofuranyl, indolyl, benzothiazolyl, benzoxazolyl, and the like.

The term “heterocyclyl” unless and otherwise specified refers to a non-aromatic monocyclic or bicyclic cycloalkyl group having 5 to 10 atoms in which 1 to 3 carbon atoms in a ring are replaced by heteroatoms (O, S or N), and are optionally benzofused or fused heteroaryl of 5-6 ring members and the heterocyclyl group can be optionally substituted with substituents for example halogen (F, Cl, Br, I), hydroxy, alkyl, alkenyl, alkynyl, cycloalkyl, acyl, aryl, alkoxy, alkaryl, cyano, nitro, oxo, carboxy, aminocarbonylamino, —C(═O)NRₓR_{y}, —OC(═O)NRₓR_{y} (wherein Rₓ and R_{y} are the same as defined earlier). Examples of heterocyclyl groups include tetrahydrofuranyl, dihydrofuranyl, dihydropyridinyl, piperidinyl, piperazinyl, dihydrobenzofuryl, azabicyclohexyl, dihydroindolyl, and the like.

“Heteroarylalkyl” refers to heteroaryl (wherein heteroaryl is same as defined earlier) linked through alkyl (wherein alkyl is the same as defined above) portion and the said alkyl portion contains from 1-6 carbon atoms.

“Heterocyclylalkyl” refers to heterocyclyl (wherein heterocyclyl is same as defined earlier) linked through alkyl (wherein alkyl is the same as defined above) portion and the said alkyl portion contains from 1-6 carbon atoms.

“Acyl” refers to —C(═O)R″ wherein R″ is selected from the group hydrogen, alkyl, cycloalkyl, aryl, alkaryl, hydroxy, alkoxy, heteroaryl, heterocyclyl, heteroarylalkyl or heterocyclylalkyl.

The term “protecting groups” is used herein to refer to known moieties which have the desirable property of preventing specific chemical reaction at a site on the molecule undergoing chemical modification intended to be left unaffected by the particular chemical modification. Also the protecting group, unless otherwise specified, may be used with groups such as hydroxy, amino, carboxy and example of such groups are found in T. W. Greene and P. G. M. Wuts, “Protective Groups in Organic Synthesis”, 2^{nd} ED, John Wiley and Sons, New York, N.Y. The species of the, for example, carboxylic protecting groups, amino protecting groups or hydroxy protecting groups employed is not critical so long as the derivatised moiety/moieties is/are stable to conditions of subsequent reactions and can be removed at the appropriate point without disrupting the remainder of the molecule.

In accordance with a second aspect, there is provided a method for treatment or prophylaxis of an animal or a human suffering from a disease or disorder of the respiratory, urinary and gastrointestinal systems, wherein the disease or disorder is mediated through muscarinic receptors. The method includes administration of at least one compound having the structure of Formula I.

In accordance with a third aspect, there is provided a method for treatment or prophylaxis of an animal or a human suffering from a disease or disorder associated with muscarinic receptors, comprising administering to a patient in need thereof, an effective amount of a muscarinic receptor antagonist compound as described above.

In accordance with a fourth aspect, there is provided a method for treatment or prophylaxis of an animal or a human suffering from a disease or disorder of the respiratory system such as bronchial asthma, chronic obstructive pulmonary disorders (COPD), pulmonary fibrosis, and the like; urinary system which induce such urinary disorders as urinary incontinence, lower urinary tract symptoms (LUTS), etc.; and gastrointestinal system such as irritable bowel syndrome, obesity, diabetes and gastrointestinal hyperkinesis with compounds as described above, wherein the disease or disorder is associated with muscarinic receptors.

In accordance with a fifth aspect, there are provided processes for preparing the compounds as described above.

The compounds described herein exhibit significant potency in terms of their activity, as determined by in vitro receptor binding and functional assays and in vivo experiments using anesthetized rabbits. The compounds that were found active in vitro were tested in vivo. Some of the compounds are potent muscarinic receptor antagonists with high affinity towards M₃ receptors. Therefore, pharmaceutical compositions for the possible treatment for the disease or disorders associated with muscarinic receptors are provided. In addition, the compounds can be administered orally or parenterally.

### DETAILED DESCRIPTION OF THE INVENTION

The compounds of the present invention may be prepared by techniques well known in the art and familiar to the average synthetic organic chemist. In addition, the compounds of the present invention may be prepared, for example, by generally following the reaction scheme(s) as depicted below.

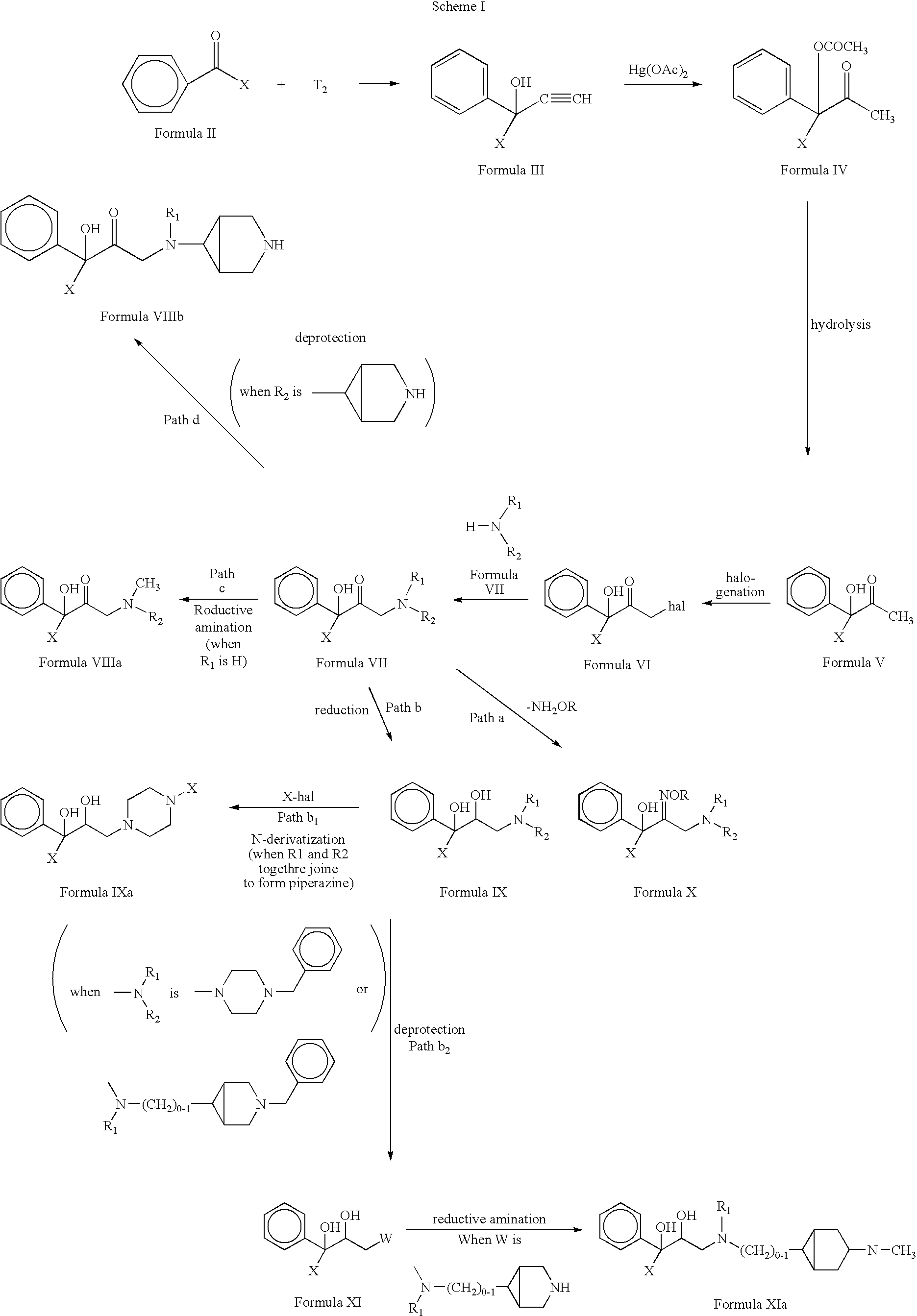

The compound of Formula VIIIb, X, IXa, XI and XIb may be prepared, for example, by the reaction sequence as shown in Scheme I. The preparation comprises reacting a compound of Formula II (wherein X is the same as defined earlier) with a compound of Formula T2 (wherein T2 is lithium acetylide, cerium acetylide, sodium acetylide, potassium acetylide or lithium acetylide in complex with diethylamine), to give a compound of Formula III, which is further reacted with mercuric acetate to give a compound of Formula IV, which is hydrolyzed to give a compound of Formula V, which is halogenated to give a compound of Formula VI (wherein hal is F, Cl, Br or I), which is reacted with a compound of Formula VII to give a compound of Formula VIII (wherein R₁ and R₂ are the same as defined earlier).
Path a: The compound of Formula VIII can be reacted with a compound of Formula —NH₂OR to give compound of Formula X (wherein R is the same as defined earlier)
Path b: The compound of Formula VIII can be reduced to give a compound of Formula IX (when R₂ is

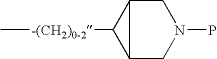

wherein P represents a protecting group such as alkaryl or acyl)
Path b1: The compound of Formula IX undergoes N-derivatization (when R1 and R2 together joins to form piperazine) to give a compound of Formula IXa.
Path b2: The compound of Formula IX undergoes deprotection to give a compound of Formula XI (wherein W is

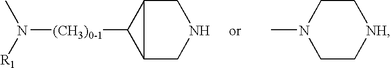

which undergoes reductive amination (when W is

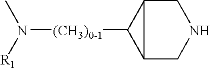

to give a compound of Formula XIa.
Path c: The compound of Formula VIII can undergo reductive amination reaction to give a compound of Formula VIIIa.
Path d: The compound of Formula VIII (when R2 is

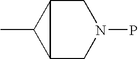

) can undergo deprotection to give a compound of Formula VIIIb.

The compound of Formula II can be reacted with a compound of Formula T2 in an organic solvent (for example, tetrahydrofuran, diethyl ether or 1,4-dioxane) to give a compound of Formula III which can be reacted with mercuric acetate in the presence of a corresponding anhydride (for example, acetic anhydride) in an organic solvent (for example, acetic acid, propionic acid or formic acid) to give a compound of Formula IV which can be hydrolyzed in the presence of an inorganic base (for example, potassium hydroxide, sodium hydroxide or lithium hydroxide) in an organic solvent (for example, methanol, ethanol, propanol or isopropanol) to give a compound of Formula V which can be halogenated in the presence of a halogenating agent (for example, pyridine hydrobromide perbromide, 2-pyrrolidone hydrobromide perbromide, N-bromosuccinimide, N-chlorosuccinimide or N-iodosuccinimide) in an organic solvent (for example, tetrahydrofuran, diethyl ether or 1,4-dioxane) to give a compound of Formula VI which can be reacted with a compound of Formula VII in the presence of an organic base (for example, triethylamine, pyridine, diisopropylamine or N-methylmorpholine in an organic solvent (for example, dichloromethane, dichloroethane, chloroform or carbon tetrachloride) to give a compound of Formula VIII which can be reacted with a compound of Formula —NH₂OR (path a) in the presence of an organic base (for example, pyridine, triethylamine or trimethylamine) in an organic solvent (for example, ethanol, methanol, propanol or isopropanol) to give a compound of Formula X. The reduction of the compound of Formula VIII (path b) can be carried out in the presence of a reducing agent (for example, sodium borohydride, sodium cyanoborohydride or lithium aluminum hydride) in an organic solvent (for example, methanol, ethanol, propanol or isopropanol) to give a compound of Formula IX.

The compound of Formula IX undergoes N-derivatization (path b1) to give a compound of Formula IXa in an organic solvent (for example, acetonitrile, dichloromethane, chloroform or carbon tetrachloride) in the presence of a base (for example, potassium carbonate, sodium carbonate or sodium bicarbonate). The compound of Formula IX undergoes (path b₂) undergo deprotection in the presence of a deprotecting agent (for example, palladium on carbon in presence of hydrogen gas or palladium on carbon in ammonium formate solution) in an organic solvent (for example, methanol, ethanol, propanol or isopropyl alcohol) to a give a compound of Formula XI, which undergoes reductive amination to give a compound of Formula XIa with formaldehyde in an organic solvent (for example, acetonitrile or dichloromethane) with formaldehyde in the presence of reducing agent (for example, sodium cyanoborohydride or sodium triacetoxy borohydride). The reductive amination of a compound of Formula VIII (path c) with formaldehyde to give a compound of Formula VIII can be carried out in an organic solvent (for example, acetonitrile or dichloromethane) with formaldehyde in the presence of reducing agent (for example, sodium cyanoborohydride or sodium triacetoxy borohydride. The compound of Formula VIII (when R₂ is

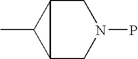

) (path d) can undergo deprotection to give a compound of Formula VIIIb in the presence of a deprotecting agent (for example, palladium on carbon in presence of hydrogen gas or palladium on carbon in ammonium formate solution) in an organic solvent (for example, methanol, ethanol, propanol or isopropyl alcohol).

Particular compounds generally prepared in this manner are shown here:
3-(3-Azabicyclo[3.1.0]hex-6-ylamino)-1-cyclopentyl-1-phenyl-propane-1,2-diol (Compound No. 3)
3-(3-Azabicyclo[3.1.0]hex-6-ylamino)-1,1-diphenyl-propane-1,2-diol (Compound No. 5)
3-(3-Benzyl-3-azabicyclo[3.1.0]hex-6-ylmethyl)-methyl-amino]-1-cyclopentyl-1-phenyl-propane-1,2-diol (Compound No. 6)
3-[(3-benzyl-3-aza-bicyclo[3.1.0]hex-6-ylmethyl)-methyl-amino]-1-cyclopentyl-1-hydroxy-1-phenyl-propan-2-one-oxime (Compound No. 8)
3-[(3-Azabicyclo[3.1.0]hex-6-ylmethyl)-methyl-amino]-1-cyclopentyl-1-phenyl-propane-1,2-diol (Compound No. 9)
1-Cyclopentyl-1-phenyl-3-(piperazin-1-yl)-propane-1,2-diol (Compound No. 10)
1-Cyclopentyl-3-(4-methyl-piperazin-1-yl)-1-phenyl-propane-1,2-diol (Compound No. 11)
3-(4-Benzyl-piperazin-1-yl)-1-cyclopentyl-1-phenyl-propane-1,2-diol (Compound No. 12)
1-Cyclopentyl-1-phenyl-3-(piperidin-1-yl)-propane-1,2-diol (Compound No. 13)
1-Cyclopentyl-1-hydroxy-1-phenyl-3-(piperidin-1-yl)-propan-2-one (Compound No. 14)
1-Cyclopentyl-1-hydroxy-3-(morpholin-4-yl)-1-phenyl-propane-2-one (Compound No. 15)
1-Cyclopentyl-3-(morpholin-4-yl)-1-phenyl-propane-1,2-diol (Compound No. 16)
3-(1-Benzyl-piperidin-4-ylamino)-1-cyclopentyl-1-hydroxy-1-phenyl-propan-2-one (Compound No. 17)
3-(1-Benzyl-piperidin-4-ylamino)-1-cyclopentyl-1-phenyl-1,2-diol (Compound No. 18)
3-[(1-Benzyl-piperidin-4-yl)-methyl-amino]-1-cyclopentyl-1-hydroxy-1-phenyl-propan-2-one (Compound No. 19)
3-(1-Benzyl-piperidin-4-ylamino)-1-cyclopentyl-1-hydroxy-1-phenyl-propan-2-one oxime (Compound No. 20)
1-Cyclopentyl-3-(3,5-dimethyl-piperazin-1-yl)-1-hydroxy-1-phenyl-propan-2-one (Compound No. 21)
1-Cyclopentyl-3-(3,5-dimethyl-piperazin-1-yl)-1-phenyl-propane-1,2-diol (Compound No. 22)
1-Cyclopentyl-1-hydroxy-3-[methyl-(1-methyl-piperidin-4-yl)-amino]-1-phenyl-propan-2-one (Compound No. 23)
1-Cyclopentyl-3-[methyl-(1-methyl-piperidin-4-yl)-amino]-1-phenyl-propane-1,2-diol (Compound No. 24)
3-[(3-Benzyl-3-aza-bicyclo[3.1.0]hex-6-yl)-methyl-amino]-1-cyclopentyl-1-hydroxy-1-phenyl-propan-2-one (Compound No. 25)
3-[(3-Benzyl-3-aza-bicyclo[3.1.0]hex-6-yl)-methyl-amino]-1-cyclopentyl-1-phenyl-propane-1,2-diol (Compound No. 26)
3-[(3-Aza-bicyclo[3.1.0]hex-6-yl)-methyl-amino]-1-cyclopentyl-1-hydroxy-1-phenyl-propan-2-one (Compound No. 27)
3-(1-Benzyl-piperidin-4-ylamino)-1-cyclopentyl-1-hydroxy-1-phenyl-propan-2-one (Compound No. 28)
3-(1-Benzyl-pyrrolidin-3-ylamino)-1-cyclopentyl-1-propan-1,2-diol (Compound No. 29)
1,1-Diphenyl-3-piperazin-1-yl-propane-1,2-diol (Compound No. 30)
3-(4-Methyl-piperazin-1-yl)-1,1-diphenyl-propane-1,2-diol (Compound No. 31)
1-Hydroxy-3-[methyl-(1-methyl-piperidin-4-yl)-amino)-1,1-diphenyl-propan-2-one (Compound No. 32)
3-[Methyl-(1-methyl-piperidin-4-yl)-amino]-1,1-diphenyl-propane-1,2-diol (Compound No. 33)
3-(1-Benzyl-pyrrolidin-3-ylamino)-1-cyclopentyl-1-hydroxy-1-phenyl-propan-2-one (Compound No. 34)
3-[(1-Benzyl-pyrrolidin-3-yl)-methyl-amino]-1-cyclopentyl-1-hydroxy-1-phenyl-propan-2-one (Compound No. 35)
3-[4-(4-Chloro-benzyl)-piperazin-1-yl]-1-cyclopentyl-1-phenyl-propane-1,2-diol (Compound No. 36)
1-Cyclopentyl-1-hydroxy-3-(3-methyl-piperazin-1-yl)-1-phenyl-propan-2-one (Compound No. 39)
1-Cyclopentyl-3-(3-methyl-piperazin-1-yl)-1-phenyl-propane-1,2-diol (Compound No. 40)
1-Cyclopentyl-3-[1,4]diazepan-1-yl-1-hydroxy-1-phenyl-propan-2-one (Compound No. 41)
1-Cyclopentyl-3-[1,4]diazepan-1-yl-1-phenyl-propane-1,2-diol (Compound No. 42)
3-[1,4′]Bipiperidinyl-1′-yl-1-cyclopentyl-1-hydroxy-1-phenyl-propan-2-one (Compound No. 43)
3-[1,4′]Bipiperidinyl-1′-yl-1-cyclopentyl-1-phenyl-propane-1,2-diol (Compound No. 44)
(2R)-1-Cyclopentyl-3-{[2-(dimethylamino)ethyl]amino}-1-hydroxy-1-phenylacetone (Compound No. 45)
1-Cyclopentyl-3-[1′-(3-cyclopentyl-2,3-dihydroxy-3-phenyl-propyl)-[4,4′]bipiperidinyl-1yl]-1-phenyl-propane-1,2-diol (Compound No. 46)
3-(1-Benzyl-pyrrolidin-3-ylamino)-1,1-diphenyl-propane-1,2-diol (Compound No. 47)
3-[4-(2-Benzo[1,3]dioxol-5-yl-ethyl)-[1,4]diazepan-1-yl]-1-cyclopentyl-1-phenyl-propane-1,2-diol (Compound No. 51)
1-Cyclopentyl-3-dimethylamino-1-hydroxy-1-phenyl-propan-2-one (Compound No. 53)
1-Cyclopentyl-3-dimethylamino-1-phenyl-propane-1,2-diol (Compound No. 54)
3-[1,4]Diazepan-1-yl-1-hydroxy-1,1-diphenyl-propan-2-one (Compound No. 55)
3-[1,4]Diazepan-1-yl-1,1-diphenyl-propane-1,2-diol (Compound No. 56)
3-[4-(2-Benzo[1,3]dioxol-5-yl-ethyl)-[1,4]diazepan-1-yl]-1-hydroxy-1,1-diphenyl-propan-2-one (Compound No. 57)
3-[4-(2-Benzo[1,3]dioxol-5-yl-ethyl)-[1,4]diazepan-1-yl]-1,1-diphenyl-propane-1,2-diol (Compound No. 58)
1-Cyclopentyl-3-(4-hydroxy-piperidin-1-yl)-1-phenyl-propane-1,2-diol (Compound No. 59)
1-Cyclopentyl-3-(2-dimethylamino-ethylamino)-1-phenyl-propane-1,2-diol (Compound No. 60)
1-Cyclopentyl-1-hydroxy-3-(4-methyl-[1,4]diazepan-1-yl)-1-phenyl-propan-2-one (Compound No. 61)
1-Cyclopentyl-3-(4-methyl-[1,4]diazepan-1-yl)-1-phenyl-propane-1,2-diol (Compound No. 62)
1-[1,4]Diazepan-1-yl-3-hydroxy-4-methyl-3-phenyl-pentan-2-one (Compound No. 63)
1-[1,4]Diazepan-1-yl-4-methyl-3-phenyl-pentane-2,3-diol (Compound No. 64)
1-Cyclopentyl-1-hydroxy-3-imidazol-1-yl-1-phenyl-propan-2-one (Compound No. 65)
1-Cyclopentyl-3-imidazol-1-yl-1-phenyl-propane-1,2-diol (Compound No. 66)
1-Cyclopentyl-1-hydroxy-3-(2-methyl-imidazol-1-yl)-1-phenyl-propan-2-one (Compound No. 67)
1-Cyclopentyl-3-(2-methyl-imidazol-1-yl)-1-phenyl-propane-1,2-diol (Compound No. 68)
1-Cyclopentyl-1-hydroxy-3-(2-isopropyl-imidazol-1-yl)-1-phenyl-propan-2-one (Compound No. 69)
1-Cyclopentyl-3-(2-isopropyl-imidazol-1-yl)-1-phenyl-propane-1,2-diol (Compound No. 70)
1-Cyclopentyl-1-hydroxy-3-(2-methyl-4,5-dihydro-imidazol-1-yl)-1-phenyl-propan-2-one (Compound No. 71)
1-Cyclopentyl-1-hydroxy-1-phenyl-3-pyrrolidin-1-yl-propan-2-one (Compound No. 72)
1-Cyclopentyl-1-phenyl-3-pyrrolidin-1-yl-propane-1,2-diol (Compound No. 73)
3-Azepan-1-yl-1-cyclopentyl-1-hydroxy-1-phenyl-propan-2-one (Compound No. 74)
3-Azepan-1-yl-1-cyclopentyl-1-phenyl-propane-1,2-diol (Compound No. 75)
1-Cyclopentyl-1-hydroxy-3-(3-hydroxy-piperidin-1-yl)-1-phenyl-propan-2-one (Compound No. 76)
1-Cyclopentyl-3-(3-hydroxy-piperidin-1-yl)-1-phenyl-propane-1,2-diol (Compound No. 77)
3-(4-Benzyl-piperidin-1-yl)-1-cyclopentyl-1-hydroxy-1-phenyl-propan-2-one (Compound No. 78)
1-Cyclopentyl-3-(2-dimethylamino-ethylamino)-1-phenyl-propane-1,2-diol (Compound No. 80)
(2R)-1-Cyclopentyl-1-hydroxy-3-(2-methyl-imidazol-1-yl)-1-phenyl-propan-2-one (Compound No. 81)
5-(3-Cyclopentyl-3-hydroxy-2-oxo-3-phenyl-propyl)-2,5-diaza-bicyclo[2.2.1]heptane-2-carboxylic acid tert-butyl ester (Compound No. 82)
Methanesulfonic acid 3-(3-cyclopentyl-3-hydroxy-2-oxo-3-phenyl-propyl)-3-aza-bicyclo[3.2.1]oct-8-yl ester (Compound No. 83)
Methanesulfonic acid 3-(3-cyclopentyl-3-hydroxy-2-oxo-3-phenyl-propyl)-3-aza-bicyclo[3.2.1]oct-8-yl ester (Compound No. 84)
(2R)-1-Cyclopentyl-1-hydroxy-3-(2-methyl-4,5-dihydro-imidazol-1-yl)-1-phenyl-propan-2-one (Compound No. 85)

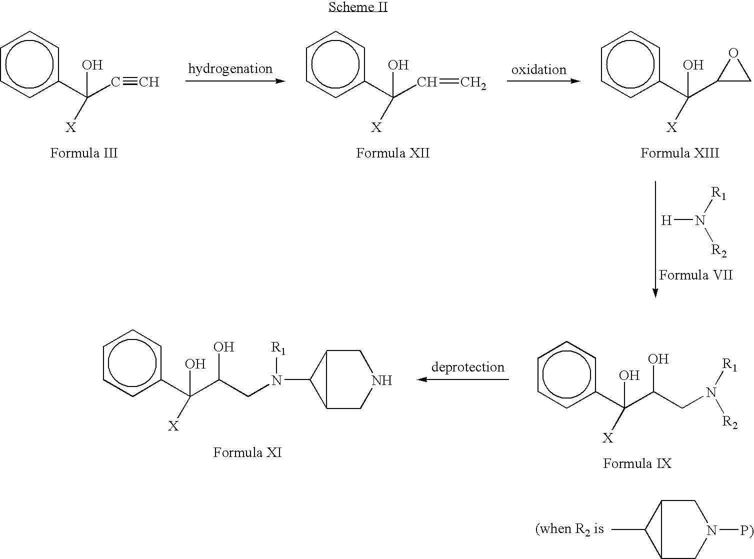

The compounds of Formula XI may also be prepared, (for example, by the reaction sequence as shown in Scheme II. The preparation comprises hydrogenating a compound of Formula III (where X is the same as defined earlier except alkyne) to give a compound of Formula XII, which can be oxidized to give a compound of Formula XIII, which can be reacted with a compound of Formula VII to give a compound of Formula IX (wherein R₁ and R₂ are the same as defined earlier), which can be deprotected (when R₂ is

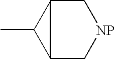

wherein P is the same as defined earlier) to give a compound of Formula XI.

Hydrogenation of a compound of Formula III can be carried out in the presence of a reducing agent (for example, palladium on calcium carbonate or sodium in liquid ammonia solution) in a hydrocarbon (for example, toluene, heptane, xylene or benzene) to give a compound of Formula XII which can be oxidized in the presence of an oxidizing agent (for example, m-chloroperbenzoic acid, perbenzoic acid or peracetic acid) in an organic solvent (for example, dichloromethane, dichloroethane, carbon tetrachloride or chloroform) to give a compound of Formula XIII which can be reacted with a compound of Formula VII in the presence of an organic base (for example, triethylamine, pyridine, N-methylmorpholine or diisopropylethylamine) in an organic solvent (for example, dichloromethane, dichloroethane, carbon tetrachloride or chloroform) to give a compound of Formula IX which can be deprotected in the presence of a deprotecting agent (for example, palladium on carbon in hydrogen gas or palladium on carbon in ammonium formate solution in an organic solvent (for example, methanol, ethyl acetate, ethanol or isopropanol) to give compound of Formula XI.

Particular exemplary compounds prepared according to the procedure described are shown here:
(R or S) 3-(3-Benzyl-3-azabicyclo[3.1.0]hex-6-ylamino)-1-cyclopentyl-1-phenyl-propane-1,2-diol (Compound No. 1)
(R or S) 3-(3-Benzyl-3-azabicyclo[3.1.0]hex-6-ylamino)-1,1-diphenyl-propane-1,2-diol (Compound No. 2)
(R or S) 3-[(3-Benzyl-3-azabicyclo[3.1.0]hex-6-ylmethyl-amino)]-1-cyclopentyl-1-phenyl-propane-1,2-diol (Compound No. 4)
(R or S) 3-(3-Benzyl-3-azabicyclo[3.1.0]hex-6-ylamino)-1-cyclopentyl-1-phenyl-propane-1,2-diol (Compound No. 7)

The compounds of Formula XI may be prepared, for example, by the reaction sequence as shown in Scheme III. The preparation comprises reacting a compound of Formula II (wherein X is the same as defined earlier) with vinyl magnesium bromide to give a compound of Formula XII, which can be oxidized to give a compound of Formula XIII, which can be reacted with a compound of Formula VII to give a compound of Formula IX (wherein R₁ and R₂ are the same as defined earlier), which can be deprotected (when R₂ is

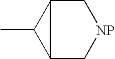

wherein P is the same as defined earlier) to give a compound of Formula XI.

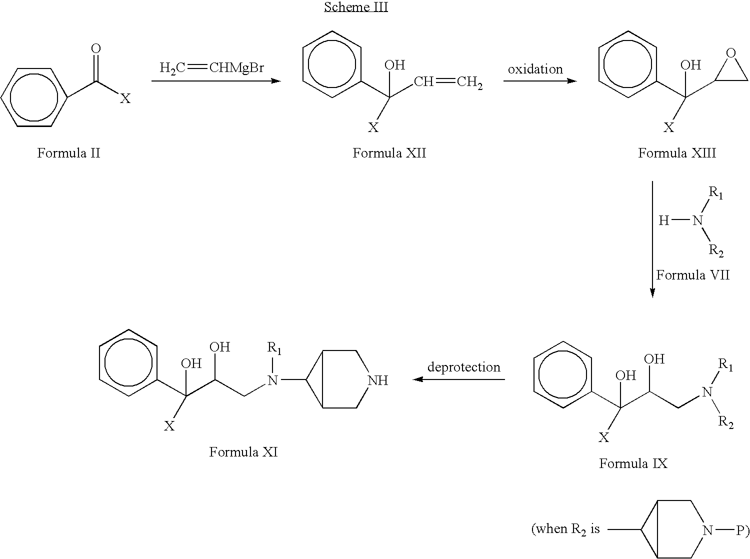

The reaction of a compound of Formula II with vinyl magnesium bromide can be carried out in an organic solvent (for example, tetrahydrofuran, diethyl ether or dioxane) to give a compound of Formula XII which can undergo oxidation in the presence of an oxidizing agent (for example, m-chloroperbenzoic acid, perbenzoic acid or peracetic acid) in an organic solvent (for example, dichloromethane, dichloroethane, carbon tetrachloride or chloroform) to give a compound of Formula XIII which on reaction with a compound of Formula VII in the presence of an organic base (for example, triethylamine, pyridine, N-methylmorpholine or di-isopropyl ethylamine in an organic solvent (for example, dichloromethane, dichloroethane, carbon tetrachloride or chloroform) can give a compound of Formula IX which can undergo deprotection in the presence of a deprotecting agent (for example, palladium on carbon or palladium on carbon) in ammonium formate solution in an organic solvent (for example, methanol, ethyl acetate, ethanol or isopropanol) to give a compound of Formula XI.

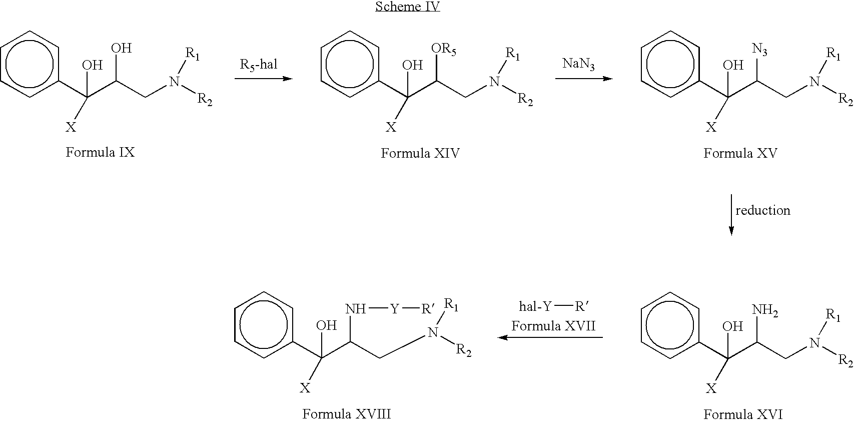

The compounds of Formula XVIII may be prepared, for example, by the reaction sequence as shown in Scheme IV. The preparation comprises reacting a compound of Formula IX (wherein X is the same as defined earlier) with a compound of Formula R₅-hal (wherein R₅ is mesyl, tosyl or 4-nitrobenzenesulphonyl group and hal is the same as defined earlier) to give of Formula XIV, which can be treated with sodium azide to give a compound of Formula XV, which can be farther reduced to give a compound of Formula XVI, which is reacted with a compound of Formula XVII to give a compound of Formula XVIII (wherein R′ and Y the same as defined earlier).

The compound of Formula IX can be reacted with a compound of Formula R₅-hal in the presence of an organic base (for example, triethyl amine or trimethyl amine) in an organic solvent (for example, dichloromethane, chloroform or carbon tetrachloride) to give a compound of Formula XIV which can be reacted with sodium azide in an organic solvent (for example, dimethylformamide or dimethylsulphoxide) to give a compound of Formula XV which can be reduced with a suitable reducing agent (for example, triphenylphosphine or lithium aluminum hydride) in an organic solvent (for example, tetrahydrofuran or 1,4-dioxane) to give a compound of Formula XVI which can be reacted with a compound of Formula XVII in the presence of an organic base (for example, triethylamine or pyridine) in an organic solvent (for example, dichloromethane, carbon tetrachloride or ethyl acetate) to give a compound of Formula XVIII.

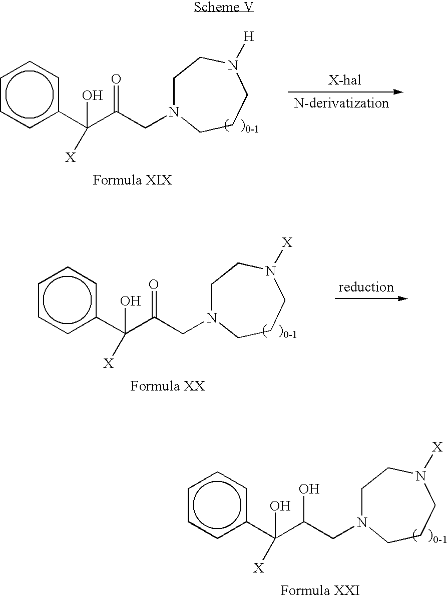

The compounds of Formulae XX and XXI may be prepared, for example, by the reaction sequence as shown in Scheme V. The preparation comprises N-derivatizing a compound of Formula XIX to give a compound of Formula XX, which undergoes reduction to give a compound of Formula XXI.

The N-derivatization of a compound of Formula XIX to give a compound of Formula XX can be carried out in an organic solvent (for example, acetonitrile, dichloromethane, chloroform or carbon tetrachloride) in the presence of a base (for example, potassium carbonate, sodium carbonate or sodium bicarbonate).

The reduction of a compound of Formula XX to give a compound of Formula XXI can be carried out in the presence of a reducing agent (for example, sodium borohydride, sodium cyanoborohydride or lithium aluminum hydride) in an organic solvent (for example, methanol, ethanol, propanol or isopropanol).

Particular exemplary compounds prepared according to the procedure described are shown here:
3-[4-(2-Benzo[1,3]dioxol-5-yl-ethyl)-piperazin-1-yl]-1-cyclopentyl-1-phenyl-propane-1,2-diol (Compound No. 37)
3-[4-(2-Benzo[1,3]dioxol-5-yl-ethyl)-piperazin-1-yl]-1-cyclopentyl-1-phenyl-propane-1,2-diol (Compound No. 38)
1-Cyclopentyl-3-[4-(3-methyl-but-2-enyl)-piperazin-1-yl]-1-phenyl-propane-1,2-diol (Compound No. 48)
3-[4-(2-Benzo[1,3]dioxol-5-yl-ethyl)-[1,4]diazepan-1-yl]-1-cyclopentyl-1hydroxy-1-phenyl-propan-2-one (Compound No. 49)
1-Cyclopentyl-3-{4-[2-(2,3-dihydro-benzofuran-5-yl)-ethyl]-[1,4]diazepan-1-yl}-1-hydroxy-1-phenyl-propan-2-one (Compound No. 50)
1-Cyclopentyl-3-{4-[2-(2,3-dihydro-benzofuran-5-yl)-ethyl]-[1,4]diazepan-1-yl}-1-phenyl-propane-1,2-diol (Compound No. 52)
3-[4-(2-Benzo[1,3]dioxol-5-yl-ethyl)-[1,4]diazepan-1-yl]-1-cyclopentyl-1-phenyl-propane-1,2-diol (Compound No. 79)

In the above scheme, where specific bases, condensing agents, protecting groups, deprotecting agents, solvents, catalysts, temperatures, etc. are mentioned, it is to be understood that other bases, condensing agents, protecting groups, deprotecting agents, solvents, catalysts, temperatures, etc. known to those skilled in the art may be used. Similarly, the reaction temperature and duration may be adjusted according to the desired needs.

Suitable salts of the compounds represented by the Formula I were prepared so as to solubilize the compound in aqueous medium for biological evaluations, as well as to be compatible with various dosage formulations and also to aid in the bioavailability of the compounds. Examples of such salts include pharmacologically acceptable salts such as inorganic acid salts (for example, hydrochloride, hydrobromide, sulphate, nitrate and phosphate), organic acid salts (for example, acetate, tartarate, citrate, fumarate, maleate, tolounesulphonate and methanesulphonate). When carboxyl groups are included in the Formula I as substituents, they may be present in the form of an alkaline or alkali metal salt (for example, sodium, potassium, calcium, magnesium, and the like). These salts may be prepared by various techniques, such as treating the compound with an equivalent amount of inorganic or organic, acid or base in a suitable solvent

TABLE 1

Formula I

| Compound | | | | | |
|---|---|---|---|---|---|
| No. | X | G | R₁ | R₂ | 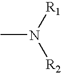 |
| 1. | 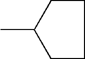 | —OH | H | 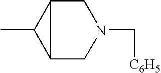 | — |
| 2. | 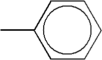 | —OH | H | 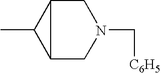 | — |
| 3. | 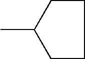 | —OH | H | | — |
| 4. | 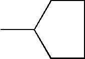 | —OH | H | 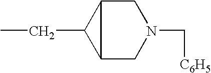 | — |
| 5. | 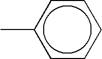 | —OH | H | 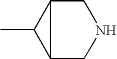 | — |
| 6. | 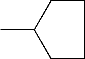 | —OH | —CH₃ | 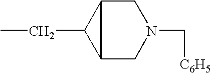 | — |
| 7. * | 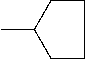 | —OH | H | 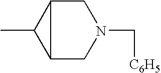 | — |
| 8. | 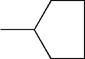 | —NOH | —CH₃ | 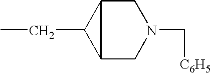 | — |
| 9 | 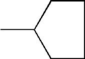 | —OH | —CH₃ | 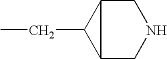 | — |
| 10. | 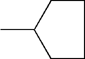 | —OH | — | — | 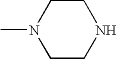 |
| 11. | 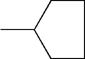 | —OH | — | — | 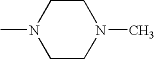 |
| 12. | 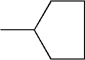 | —OH | — | — | 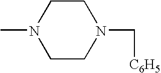 |
| 13. | 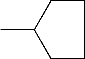 | —OH | — | — | 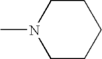 |
| 14. | 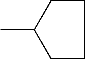 | —O | — | — | 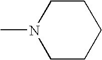 |
| 15. | 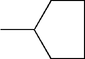 | —O | — | — | 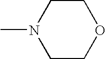 |
| 16. | 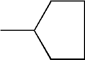 | —OH | — | — | 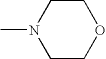 |
| 17. | 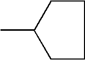 | —O | H | 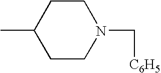 | — |
| 18 | 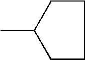 | —OH | H | 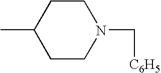 | — |
| 19. | 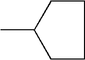 | —O | —CH₃ | 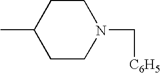 | — |
| 20. | 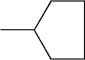 | NOH | H | 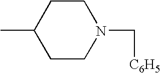 | — |
| 21. | 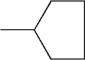 | —O | — | — | 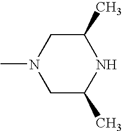 |
| 22. | 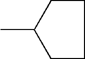 | —OH | — | — | 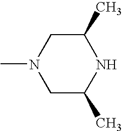 |
| 23. | 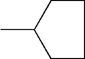 | —O | —CH₃ | 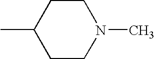 | — |
| 24. | 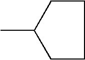 | —OH | —CH₃ | 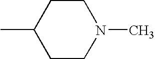 | — |
| 25. | 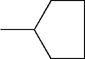 | —O | —CH₃ | 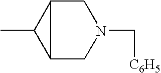 | — |
| 26. | 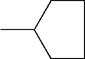 | —OH | —CH₃ | 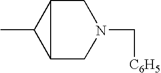 | — |
| 27. | 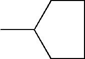 | —O | —CH₃ | 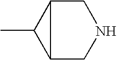 | — |
| 28. | 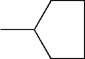 | —O | H | 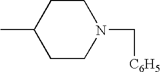 | — |
| 29. | 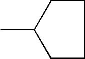 | —OH | H | 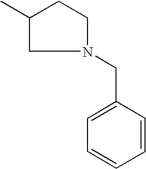 | — |
| 30. | 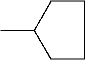 | —OH | — | — | 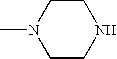 |
| 31. | 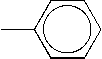 | —OH | — | — | 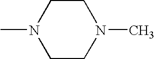 |
| 32. | 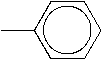 | —O | —CH₃ | 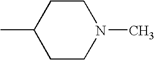 | — |
| 33. | 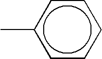 | —OH | —CH₃ | 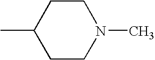 | — |
| 34. | 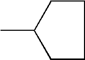 | —O | H | 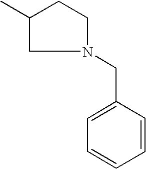 | — |
| 35. | 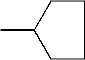 | —O | —CH₃ | 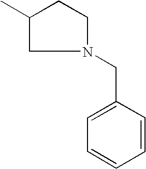 | — |
| 36. | 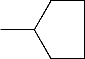 | —OH | — | — | 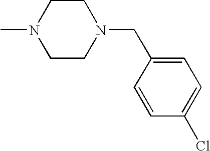 |
| 37. | 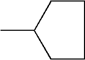 | —OH | — | — | 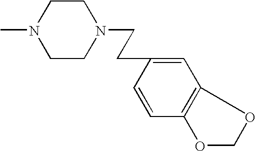 |
| 38. | 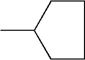 | —OH | — | — | 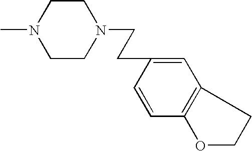 |
| 39. | 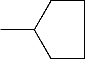 | —O | — | — | 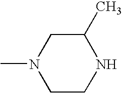 |
| 40. | 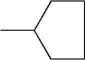 | —OH | — | — | 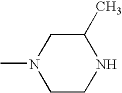 |
| 41. | 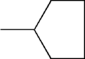 | —O | — | — | 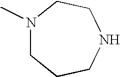 |
| 42. | 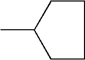 | —OH | — | — | 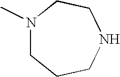 |
| 43. | 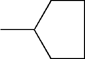 | —O | — | — | 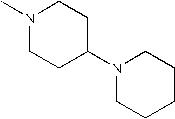 |
| 44. | 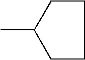 | —OH | — | — | 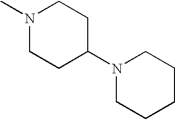 |
| 45. | 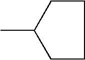 | —O | H | —CH₂CH₂N(CH₃)₂ | — |
| 46. | 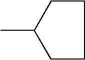 | —OH | — | — | 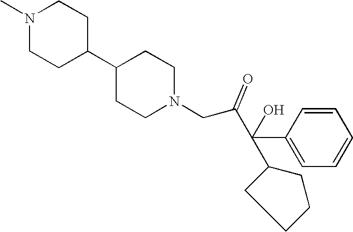 |
| 47. | 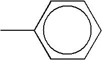 | —OH | H | 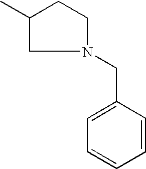 | — |
| 48. | 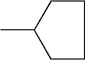 | —OH | — | — | 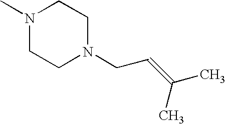 |
| 49. | 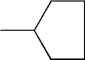 | —O | — | — | 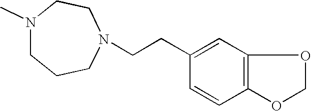 |
| 50. | 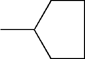 | —O | — | — | 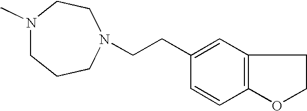 |
| 51. | 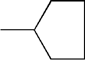 | —OH | — | — | 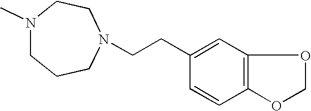 |
| 52. | 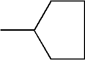 | —OH | — | — | 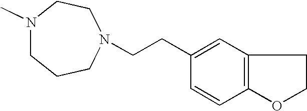 |
| 53. | 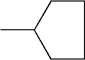 | —O | —CH₃ | —CH₃ | — |
| 54. | 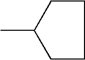 | —OH | CH₃ | —CH₃ | — |
| 55. | 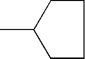 | —O | — | — | 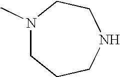 |
| 56. | 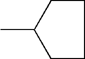 | —OH | — | — | 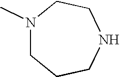 |
| 57. | 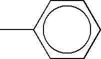 | —O | — | — | 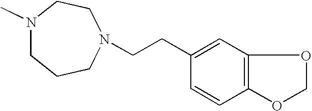 |
| 58. | 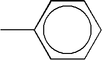 | —OH | — | — | 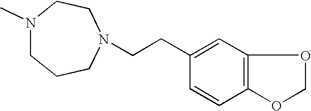 |
| 59. | 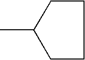 | —OH | — | — | 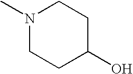 |
| 60. | 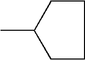 | —OH | H | —CH₂CH₂N(CH₃)₂ | — |
| 61. | 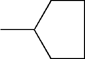 | —O | — | — | 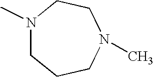 |
| 62. | 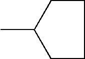 | —OH | — | — | 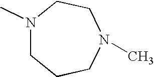 |
| 63. | 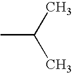 | —O | — | — | 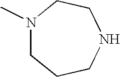 |
| 64. | 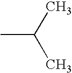 | —OH | — | — | 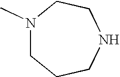 |
| 65. | 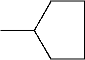 | —O | — | — | 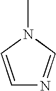 |
| 66. | 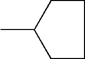 | —OH | — | — | 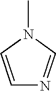 |
| 67. | 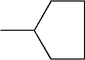 | —O | — | — | 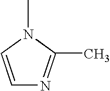 |
| 68. | 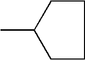 | —OH | — | — | 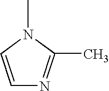 |
| 69. | 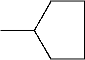 | —O | — | — | 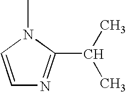 |
| 70. | 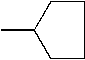 | —OH | — | — | 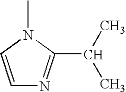 |
| 71. | 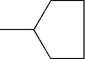 | —O | — | — | 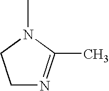 |
| 72. | 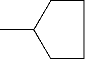 | —O | — | — | 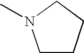 |
| 73. | 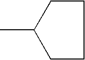 | —OH | — | — | |
| | 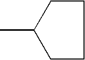 | —O | — | — | 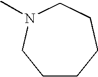 |
| 75. | 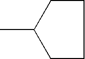 | —OH | — | — | 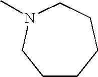 |
| 76. | 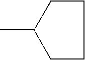 | —O | — | — | 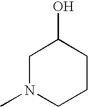 |
| 77. | 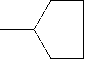 | —OH | — | — | 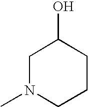 |
| 78. | 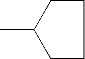 | —O | — | — | 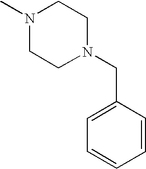 |
| 79. | 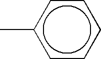 | —O | — | — | 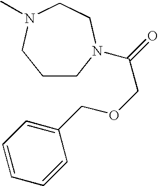 |
| 80. & | 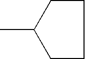 | —OH | H | —CH₂CH₂N(CH₃)₂ | — |
| 81. && | 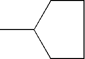 | —O | — | — | 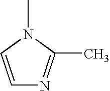 |
| 82. | 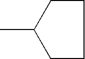 | —O | — | — | 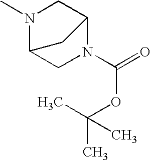 |
| 83. | 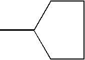 | —O | — | — | 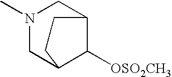 |
| 84. | 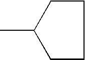 | —O | — | — | 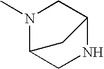 |
| 85. &&& | 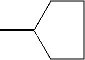 | —O | — | — | 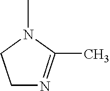 |

* represents an isomer of compound No. 1
& represents R isomer of Compound No. 60
&& represents R isomer of Compound No. 67
&&& represents R isomer of Compound No. 71

Because of their valuable pharmacological properties, the compounds described herein may be administered to an animal for treatment orally, or by a parenteral route. The pharmaceutical compositions described herein can be produced and administered in dosage units, each unit containing a certain amount of at least one compound described herein and/or at least one physiologically acceptable addition salt thereof. The dosage may be varied over extremely wide limits, as the compounds are effective at low dosage levels and relatively free of toxicity. The compounds may be administered in the low micromolar concentration, which is therapeutically effective, and the dosage may be increased as desired up to the maximum dosage tolerated by the patient.

The compounds described herein can be produced and formulated as their enantiomers, diastereomers, N-Oxides, polymorphs, solvates and pharmaceutically acceptable salts, as well as metabolites having the same type of activity. Pharmaceutical compositions comprising the molecules of Formula I or metabolites, enantiomers, diastereomers, N-oxides, polymorphs, solvates or pharmaceutically acceptable salts thereof, in combination with pharmaceutically acceptable carrier and optionally included excipient can also be produced.

The examples mentioned below demonstrate general synthetic procedures, as well as specific preparations of particular compounds. The examples are provided to illustrate the details of the invention and do not limit the scope of the present invention.

### EXAMPLES

Various solvents, such as acetone, methanol, pyridine, ether, tetrahydrofuran, hexanes, and dichloromethane, were dried using various drying reagents according to procedures described in the literature. IR spectra were recorded as Nujol mulls or a thin neat film on a Perkin Elmer Paragon instrument. Nuclear Magnetic Resonance (NMR) spectra were recorded on a Varian XL-300 MHz instrument using tetramethylsilane as an internal standard.

### Example 1

### Preparation of 1-cyclopentyl-1-hydroxy-1-phenyl propyne

A solution of n-butyl lithium (15%, 54.0 mL, 92.0 mM) in dry tetrahydrofuran (200.0 mL) was saturated with dry acetylene gas at −78° C. To the reaction mixture thus obtained was added phenyl cyclopentyl ketone (8.0 g, 46.0 mM) in dry tetrahydrofuran (50.0 mL) dropwise at the same temperature under stirring. The reaction mixture was brought to 25-30° C. under stirring and saturated ammonium chloride solution (30.0 mL) was added, followed by stirring for 5 minutes. The organic layer was separated and washed with saturated brine solution (30.0 mL). Tetrahydrofuran was distilled off under reduced pressure and the residue was purified through column chromatography using 10% ethyl acetate in hexane solvent mixture as an eluent to get the title organic compound.

Yield: 87% (8.0 g); IR (DCM): 3422.8, 3299.9, 2110.9 cm⁻¹; ¹H NMR (CDCl₃): δ 7.65-7.67 (m, 2H), 7.28-7.39 (m, 3H), 2.68 (s, 1H), s, 1H), 2.38-2.43 (m, 1H), 1.57-1.78 (m, 5H), 1.40-1.50 (m, 3H).

### Example 2

### Preparation of 1-cyclopentyl-1-acetoxy-1-phenyl-2-propanone

To a solution of 1-cyclopentyl-1-hydroxy-1-phenyl-2-propyne (3.0 g, 15 mM) in glacial acetic acid (21.0 mL) was added acetic anhydride (2.1 mL) followed by addition of mercuric acetate (5.3 g, 16.5 mM) portionwise. The reaction mixture was stirred for 72 hours at 25-30° C. followed by addition of thioacetamide (1.2 g, 16.5 mM). Stirring was continued for three hours at the same temperature. The reaction mixture was diluted with ether (150.0 mL). The reaction mixture was filtered through celite pad. The filtrate was washed with water, saturated sodium bicarbonate solution, dried over anhydrous sodium sulphate and concentrated under reduced pressure to furnish title organic compound.

Yield: 82% (3.2 g); IR (DCM): 1743.1, 1720.4 cm⁻¹; ¹H NMR (CDCl₃): δ 7.27-7.38 (m, 5H), 2.94-3.04 (m, 1H), 2.27 (s, 3H), 1.92 (s, 3H), 1.27-1.70 (m, 8H).

### Example 3

### Preparation of 1-cyclopentyl-1-hydroxy-1-phenyl-2-propanone

To a solution of 1-cyclopentyl-1-acetoxy-1-phenyl-2-propanone (4.0 g, 15.4 mM) in methanol (20.0 mL), aqueous potassium hydroxide solution (1.3 g, 23.0 mM, 1.5 mL of water) was added and the reaction mixture was refluxed for 2 hours. Cooled the reaction mixture to room temperature and methanol was removed under reduced pressure. The residue thus obtained was diluted with water (10.0 mL) and extracted with ethyl acetate (3×25.0 mL). The ethyl acetate layer was washed with water and brine solution. Dried over anhydrous sodium sulphate and concentrated under reduced pressure to get the title organic compound.

Yield: 90% (3.2 g); IR (DCM): 3456.6, 1704.5 cm⁻¹; ¹H NMR (CDCl₃): δ 7.52-7.54 (m, 2H), 7.27-7.38 (m, 3H), 4.61 (s, 1H, OH), 3.02-3.07 (m, 1H), 2.11 (s, 3H), 1.26-1.72 (m, 8H).

### Example 4

### Preparation of 3-bromo-1-cyclopentyl-1-hydroxy-1-phenyl-2-propanone

To a solution of 1-cyclopentyl-1-hydroxy-1-phenyl-2-propanone (3.4 g, 15.6 mM) in dry tetrahydrofuran (150.0 mL), tetrahydrofuran solution of pyridine hydrobromide perbromide (6.0 g, 18.7 mM, 85% pure, 100.0 mL of dry tetrahydrofuran) was added dropwise at room temperature and the stirring was continued for 36 hour at the same temperature. The solid so separated was filtered and the filtrate was concentrated under reduced pressure. The residue was purified through column chromatography using 2% ethyl acetate in hexane as an eluent to get the title organic compound.

Yield: 64% (3.0 g); IR (DCM): 1721.5 cm⁻¹; ¹H NMR (CDCl₃): δ 7.50-7.53 (m, 2H), 7.28-7.40 (m, 3H), 4.05-4.2 (m, 2H), 3.80 (s, 1H, —OH), 3.02-3.08 (m, 1H), 1.28-1.43 (m, 8H).

### Example 5

### Preparation of 1-cyclopentyl-1-hydroxy-1-phenyl-2-propene

To a solution of 1-cyclopentyl-1-hydroxy-1-phenyl-2-propyne (5.0 g, 24.8 mM) in toluene (50.0 mL), palladium on calcium carbonate (0.5 g) was added and the reaction mixture was subjected to hydrogenation at room temperature under atmospheric pressure. The reaction mixture was filtered and the residue thus obtained was purified through column chromatography using 2% ethyl acetate in hexane as an eluent to get the title organic compound.

IR (DCM): 3479.9 cm⁻¹; ¹H NMR (CDCl₃):δ 7.19-7.47 (m, 5H), 6.21-6.31 (m, 1H), 5.12-5.31 (m, 2H), 2.47-2.58 (m, 1H), 1.36-1.57 (m, 8H).

### Example 6

### Preparation of 1-cyclopentyl-1-hydroxy-1-phenyl-2,3-epoxy propane

To a solution of 1-cyclopentyl-1-hydroxy-1-phenyl-2-propene (0.95 g, 4.7 mM) in dry dichloromethane (10.0 mL) at 0-5° C., dichloromethane solution of m-chloroperbenzoic acid (2.03 g, 5.9 mM, 15.0 mL dichloromethane) was added dropwise. The reaction mixture was stirred at room temperature for 12 hour and triethylamine (3.0 mL) was added to the reaction mixture and stirred for 15 minutes. The reaction mixture was poured onto saturated sodium bicarbonate solution (10.0 mL) and dichloromethane layer was separated, washed with saturated sodium bicarbonate solution (10.0 mL), water (10.0 mL) and brine solution. Dried over anhydrous sodium sulphate and concentrated under reduced pressure. The residue was purified by column chromatography using 5% ethyl acetate in hexane to get non-polar epoxide-A (0.28 g) and polar epoxide-B (0.5 g).

Non polar epoxide-A: IR (KBr): 3477.5 cm⁻¹; ¹H NMR (CDCl₃): δ 7.18-7.46 (m, 5H), 3.36-3.38 (m, 1H), 2.57-2.63 (m, 2H), 1.32-1.67 (m, 9H).

Polar epoxide-B: IR (KBr): 3390.7 cm⁻¹; ¹H NMR (CDCl₃): δ 7.24-7.50 (m, 5H), 3.48-3.50 (m, 1H), 2.97-2.99 (m, 1H), 2.83-2.86 (m, 1H), 2.50 (m, 1H), 1.33-1.71 (m, 8H).

### Example 7

### Preparation of 1,1-diphenyl-1-hydroxy-2-propyne

The title compound was prepared by using benzophenone in place of phenyl cyclopentyl ketone following the procedure mentioned for the synthesis of 1-cyclopentyl-1-hydroxy-1-phenyl-2-propyne.

IR (DCM): 3540.0, 3438.4 cm⁻¹; ¹H NMR (CDCl₃): δ 7.59-7.62 (m, 4H), 7.27-7.36 (m, 6H), 2.89 (s, 1H), 2.86 (s, 1H).

### Example 8

### Preparation of 1,1-diphenyl-1-hydroxy-2-propene

The title compound was prepared by using corresponding acetylenic compound following the procedure mentioned for the synthesis of 1-cyclopentyl-1-hydroxy-1-phenyl-2-propene.

Yield: 90%; IR (DCM): 3449.4 cm⁻¹; ¹H NMR (CDCl₃): δ 7.24-7.43 (m, 10H), 6.47-6.56 (m, 1H), 5.30-5.35 (m, 2H).

### Example 9

### Preparation of 1,1-diphenyl-1-hydroxy-2,3-epoxy-propane

The title compound was a prepared starting from the corresponding olefin following the procedure mentioned for the synthesis of 1-cyclopentyl-1-hydroxy-1-phenyl-2,3-epoxy propane.

Polar epoxide B: Yield: 69%; IR (KBr): 3382.9 cm⁻¹; ¹H NMR (CDCl₃): δ 7.48-7.50 (m, 2H), 7.24-7.38 (m, 8H), 3.78-3.80 (m, 1H), 2.97-2.99 (m, 1H), 2.77-2.80 (m, 1H) 2.55 (s, 1H).

### Example 10

### Preparation of (1α, 5α, 6α)-6-methylamino-methyl-3-benzyl-3-azabicyclo[3.1.0]hexane

### Step a: Preparation of (1α, 5α, 6α)-6-hydroxymethyl-3-benzyl-3-azabicyclo[3.1.0]hex-6-yl)-methanolane

The organic compound was prepared following the standard protocol described in Synlett., (1996), p 1097 by using N-benzyl maleimide.

### Step b: Preparation of (1α, 5α, 6α)-6-methylsulphonyloxymethyl-3-benzyl-3-azabicyclo[3.1.0]hex-6-yl)-methanolane

To a solution compound obtained from step a above (25.6 mM) in dichloromethane at 0° C., was added triethylamine (76.8 mM) followed by the addition of methane sulphonyl chloride (51.2 mM) dropwise. The reaction mixture was cooled to 0° C. and again methane sulphonyl chloride was added. The reaction mixture was gradually warmed to room temperature and stirred for overnight. The reaction mixture was quenched by addition of water. The organic layer was separated to give the crude organic compound which was purified by column chromatography using 2-5% methanol in dichloromethane and 2% triethylamine as an eluent.

### Step c: Preparation of (1α, 5α, 6α)-6-methylamino-methyl-3-benzyl-3-azabicyclo[3.1.0]hexane

To a solution of a compound obtained from step b above (8.71 mM) in methanol (20 mL) was added methylamine solution (40%, 25 mL). The reaction mixture was heated at 85-90° C. for overnight in an autoclave followed by cooling it down to −78° C. and autoclave was opened. The solvent was evaporated off and diluted with water, dilute hydrochloric acid and ethylacetate organic layer was separated and the aqueous layer was basified with 10% of aqueous sodium hydroxide solution. The organic compound was extracted with dichloromethane and dried with sodium sulphate, evaporated to give the title organic compound.

¹H NMR (CDCl₃):δ 7.30-7.18 (5H, m), 3.57 (2H, s), 2.97-2.95 (2H, m), 2.42-2.23 (7H, m), 1.68-1.66 (1H, m), 1.19-1.17 (2H, m).

### Scheme 1, Procedure

### Example 11

### Preparation of 1-cyclopentyl-1-hydroxy-1-phenyl-3-(piperidin-1-yl)-propan-2-one (Compound No. 14)

To a solution of piperidine (0.56 g, 6.6 mM) and triethylamine (0.36 ml, 2.6 mM) in dry dichloromethane (4.0 mL), 3-bromo-1-cyclopentyl-1-hydroxy-1-phenyl-2-propanone (0.39 g, 1.3 mM) was added and the reaction mixture was stirred at room temperature for 12 hours. Dichloromethane was concentrated under reduced pressure and the residue was purified in silica gel column chromatography using 5% ethyl acetate in hexane solvent mixture as an eluent to get the title organic compound.

Yield: 86% (0.343 g); IR (DCM): 1714.0 cm⁻¹; ¹N NMR (CDCl₃):δ 7.59-7.61 (m, 2H), 7.20-7.34 (m, 3H), 3.09-3.29 (m, 2H), 2.86 (m, 1H), 2.33 (brs, 2H), 2.10-2.14 (m, 2H), 1.28-1.62 (m, 14H); Mass (m/z): 302(M⁺+1).

Analogues of 1-cyclopentyl-1-hydroxy-1-phenyl-3-piperidin-1-yl-propan-2-one (Compound No. 14) described below can be prepared by replacing the appropriate amine in place of piperidine, as applicable in each case.

1-Cyclopentyl-1-hydroxy-3-(morpholin-4-yl)-1-phenyl-propan-2-one (Compound No. 15)

IR (DCM): 1713.9 cm⁻¹; ¹H NMR (CDCl₃): δ 7.54-7.57 (m, 2H), 7.22-7.36 (m, 3H), 3.59-3.62 (m, 4H), 3.17-3.39 (m, 2H), 2.90 (m, 1H), 2.38-2.42 (m, 2H), 2.16-2.22 (m, 2H), 1.26-1.59 (m, 8H); Mass (m/z): 304 (M⁺+1).

3-(1-benzyl-piperidin-4-ylamino)-1-cyclopentyl-1-hydroxy-1-phenyl-propan-2-one (Compound No. 17)

IR (KBr): 3418.7 cm⁻¹; ¹H NMR (CDCl₃): δ 7.14-7.52 (m, 10H), 3.38-3.56 (m, 3H), 2.77-2.84 (m, 4H), 2.35-2.40 (m, 4H), 2.35-2.40 (m, 4H), 1.26-2.04 (m, 11H); Mass (m/z): 407(M⁺+1).

3-[(1-Benzyl-piperidin-4-yl)-methyl-amino]-1-cyclopentyl-1-hydroxy-1-phenyl-propan-2-one (Compound No. 19)

IR (DCM): 1715.0 cm⁻¹, 2856.3 cm⁻¹

¹H NMR (CDCl₃): δ 1.274-1.692 (m, 14H), 1.781-1.871 (m, 3H), 2.056-2.301 (m, 2H), 2.839-2.893 (m, 3H), 3.317-3.476 (m, 4H), 7.244-7.354 (m, 8H), 7.571-7.597 (m, 2H). Mass: (m/z): 421.0(M⁺+1).

1-Cyclopentyl-3-(3,5-dimethyl-piperazin-1-yl)-1-hydroxy-1-phenyl-propan-2-one (Compound No. 21)

Mass: (m/z): 331.0(M⁺+1). IR (DCM): 1713 cm⁻¹, 2958.9 cm^{-1 1}H NMR (CDCl₃): δ 1.03-1.28 (m, 3H), 1.33-1.49 (m, 3H), 1.62-1.98 (m, 12H), 2.76-2.87 (m, 3H), 3.09-3.14 (m, 2H), 3.33-3.38 (m, 2H), 7.21-7.58 (m, 5H).

1-Cyclopentyl-1-hydroxy-3-[methyl-(1-methyl-piperidin-4-yl)-amino]-1-phenyl-propan-2-one (Compound No. 23)

Mass: (m/z): 345.0(M⁺+1). IR (DCM): 1713.5 cm⁻¹, 3441.0 cm^{-1 1}H NMR (CDCl₃): δ 1.25-1.68 (m, 14H), 2.01-2.10 (m, 3H), 2.29-2.33 (m, 3H), 2.84-2.87 (m, 2H), 3.03-3.05 (m, 5H), 7.24-7.35 (m, 3H), 7.55-7.58 (m, 2H).

3-[(3-Benzyl-3-aza-bicyclo[3.1.0]hex-6-yl)-methyl-amino]-1-cyclopentyl-1-hydroxy-1-phenyl-propan-2-one (Compound No. 25)

Mass (m/z): 419 (M⁺+1) ¹H NMR (CDCl₃, 300 MHz): 1.25-1.31 (m, 3H), 1.44-1.55 (m, 8H), 2.094 (s, 3H), 2.168 (brs, 2H), 2.31-2.35 (m, 2H), 2.83-2.92 (m, 3H), 3.36-3.39 (m, 2H), 3.52 (brs, 2H), 4.73 (s, 1H), 4.88-4.89 (brs, 1H), 7.22-7.33 (m, 8H), 7.50-7.53 (m, 2H).

3-(1-Benzyl-piperidin-4-ylamino)-1-cyclopentyl-1-hydroxy-1-phenyl-propan-2-one (Compound No. 28)

Mass: (m/z): 407.0 (M⁺+1). IR (DCM): 1710.7 cm⁻¹, 2941.6 cm^{-1 1}H NMR (CDCl₃): δ 1.40-2.04 (m, 12H), 2.35-2.40 (m, 4H), 2.77-2.84 (m, 4H), 3.38-3.56 (m, 4H), 7.14-7.52 (m, 10H).

1-Hydroxy-3-[methyl-(1-methyl-piperidin-4-yl)-amino)-1,1-diphenyl-propan-2-one (Compound No. 32)

Mass: (m/z): 353.0 (M⁺+1) IR (DCM): 1720.2 cm⁻¹, 2918.3 cm^{-1 1}H NMR (CDCl₃): δ 1.255-1.284 (m, 2H), 1.555-1.623 (m, 4H), 1.834-1.908 (m, 2H), 2.174-2.218 (m, 3H), 2.272-2.343 (m, 3H), 2.805-2.923 (m, 2H), 3.473-3.537 (m, 2H), 7.224-7.511 (m, 10H).

3-(1-Benzyl-pyrrolidin-3-ylamino)-1-cyclopentyl-1-hydroxy-1-phenyl-propan-2-one (Compound No. 34)

Mass: (m/z): 393 (M⁺+1) ¹H NMR (CDCl₃, 300 MHz): 1.33-1.54 (m, 9H), 1.80-1.95 (m, 1H), 2.28-2.33 (m, 2H), 2.49-2.56 (m, 4H), 2.94-3.0 (m, 2H), 3.50-3.52 (m, 2H), 3.59-3.61 (m, 2H), 7.24-7.34 (m, 8H), 7.48-7.50 (m, 2H). IR in DCM: 3423.2, 2955.0, 2866.7, 2796.7, 1713.6

1-Cyclopentyl-1-hydroxy-3-(3-methyl-piperazin-1-yl)-1-phenyl-propan-2-one (Compound No. 39)

Mass: (m/z): 317.0(M⁺+1) IR (DCM): 1647.7 cm⁻¹, 3445.3 cm^{-1 1}H NMR (CDCl₃): δ 1.072-1.093 (m, 3H), 1.260-1.426 (m, 6H), 1.572-1.624 (m, 6H), 2.775-2.939 (m, 6H), 3.172-3.207 (m, 2H), 3.16-3.352 (m, 2H), 7.244-7.577 (m, 5H).

1-Cyclopentyl-3-[1,4]diazepan-1-yl-1-hydroxy-1-phenyl-propan-2-one (Compound No. 41)

Mass: (m/z): 317.0 (M⁺+1) IR (DCM): 1646.5 cm^{-1 1}H NMR (CDCl₃): δ 1.26-1.33 (m, 5H), 1.47-1.72 (m, 7H), 2.54-2.65 (m, 4H), 2.84-3.00 (m, 5H), 3.37-3.48 (m, 2H), 7.22-7.57 (m, 5H).

3-[1,4′]Bipiperidinyl-1′-yl-1-cyclopentyl-1-hydroxy-1-phenyl-propan-2-one (Compound No. 43)

Mol. w.t.: 386

Mass (m/z): 387 (M⁺+1) 1H NMR (CDCl₃): 1.22-1.78 (m, 19H), 1.99-2.03 (m, 2H), 2.16-2.28 (m, 2H), 2.97-2.49 (m, 4H), 2.60-2.69 (m, 2H), 3.08-3.12 (m, 1H), 4.00-4.05 (m, 1H), 7.22-7.56 (m, 5H). IR (DCM): 1448, 1638, 2938, 3425.

(2R)-1-Cyclopentyl-3-{[2-(dimethylamino)ethyl]amino}-1-hydroxy-1-phenylacetone (Compound No. 45)

1-Cyclopentyl-3-dimethylamino-1-hydroxy-1-phenyl-propan-2-one (Compound No. 53)

Mass: (m/z): 262.0 (M⁺+1) IR (DCM): 1715.8 cm⁻¹, 2955.6 cm^{-1 1}H NMR (CDCl₃): δ 1.408-1.645 (m, 9H), 2.052-2.147 (m, 6H), 2.865-2.892 (m, 1H), 3.162-3.301 (m, 2H), 7.220-7.580 (m, 5H).

3-[1,4]Diazepan-1-yl-1-hydroxy-1,1-diphenyl-propan-2-one (Compound No. 55)

Mass (m/z): 325 (M⁺+1) Melting Point: 116-122° C. ¹H NMR (CDCl₃, 300 MHz): 1.55-1.62 (m, 2H), 2.60-2.74 (m, 6H), 2.85-2.89 (m, 2H), 3.53-3.61 (s, 2H), 7.26-7.47 (m, 10H). IR in DCM: 3446.6, 3253.0, 3059.7, 2941.5, 1726.9

3-[4-(2-Benzo[1,3]dioxol-5-yl-ethyl)-[1,4]diazepan-1-yl]-1-hydroxy-1,1-diphenyl-propan-2-one (Compound No. 57)

Mass (m/z): 473 (M⁺+1) 1H NMR (CDCl₃, 300 MHz): 1.89 (brs, 2H), 2.2-2.45 (brs, 1H), 2.68-2.72 (m, 5H), 2.77-2.83 (m, 6H), 2.98 (brs, 2H), 3.66 (s, 2H), 5.92 (s, 2H), 6.60-6.74 (m, 2H), 7.26-7.43 (m, 10H).

1-Cyclopentyl-1-hydroxy-3-(4-methyl-[1,4]diazepan-1-yl)-1-phenyl-propan-2-one (Compound No. 61)

Mass: (m/z): 331.0 (M⁺+1) IR (DCM): 1713.0 cm⁻¹, 2947.5 cm^{-1 1}H NMR (CDCl₃): δ 1.262-1.428 (m, 8H), 1.565-1.731 (m, 4H), 1.936-2.367 (m, 4H), 2.567-2.675 (m, 6H), 2.866-2.891 (m, 1H), 3.329-3.477 (m, 2H), 7.242-7.583 (m, 5H).

1-[1,4]Diazepan-1-yl-3-hydroxy-4-methyl-3-phenyl-pentan-2-one (Compound No. 63)

Mass: (m/z): 293 (M⁺+1) ¹H NMR (CDCl₃, 300 MHz): 0.64-0.66 (m, 3H), 0.96-0.98 (m, 3H), 1.63-1.66 (brs, 2H), 1.68-2.20 (brs, 2H), 2.44-2.63 (m, 5H), 2.76-2.79 (m, 2H), 2.89-2.93 (m, 2H), 3.34-3.48 (m, 2H), 7.21-7.35 (m, 3H), 7.57-7.59 (m, 2H). IR in DCM: 3381.0, 3060.3, 2966.1, 1714.6

1-Cyclopentyl-1-hydroxy-3-imidazol-1-yl-1-phenyl-propan-2-one (Compound No. 65)

Mass: (m/z): 285.0 (M⁺+1) IR (DCM): 1729.6 cm⁻¹, 2956.8 cm^{-1 1}H NMR (CDCl₃): δ 1.256-1.651 (m, 9H), 3.033-3.095 (m, 1H), 4.829-4.983 (m, 2H), 6.628-7.559 (m, 8H).

1-Cyclopentyl-1-hydroxy-3-(2-methyl-imidazol-1-yl)-1-phenyl-propan-2-one (Compound No. 67)

Mass: (m/z): 299.0 (M⁺+1) IR (DCM): 1733.3 cm⁻¹, 2957.3 cm^{-1 1}H NMR (CDCl₃): δ 1.36-1.63 (m, 4H), 1.75-1.94 (m, 8H), 3.00-3.03 (m, 1H), 4.67-4.84 (m, 2H), 6.46-7.49 (m, 7H).

1-Cyclopentyl-1-hydroxy-3-(2-isopropyl-imidazol-1-yl)-1-phenyl-propan-2-one (Compound No. 69)

Mass: (m/z): 327.0 (M⁺+1) IR (DCM): 1737.1 cm⁻¹, 3446.9 cm^{-1 1}H NMR (CDCl₃): δ 1.24-1.27 (m, 6H), 1.32-1.48 (m, 4H), 1.53-1.68 (m, 5H), 2.18-2.23 (m, 1H), 3.08-3.13 (m, 1H), 4.75-4.92 (m, 2H), 6.45-7.56 (m, 7H). m.p: 157.9°-158.8° C.

1-Cyclopentyl-1-hydroxy-3-(2-methyl-4,5-dihydro-imidazol-1-yl)-1-phenyl-propan-2-one (Compound No. 71)

Mass: (m/z): 301.0 (M⁺+1) IR (DCM): 1734.9 cm⁻¹, 2923.1 cm^{-1 1}H NMR (CDCl₃): δ 1.415-1.426 (m, 4H), 2.31 (s, 3H), 2.606-2.631 (m, 1H), 2.806-2.955 (m, 2H), 3.543-3.614 (m, 2H), 3.811-3.877 (m, 2H), 4.336-4.399 (m, 1H), 5.118-5.181 (m, 2H), 7.092-7.543 (m, 7H).

1-Cyclopentyl-1-hydroxy-1-phenyl-3-pyrrolidin-1-yl-propan-2-one (Compound No.72)

Mass: (m/z): 288 (M⁺+1) ¹H NMR (CDCl₃): 1.25-1.72 (m, 12H), 2.30-2.49 (m, 4H), 2.84-2.90 (m, 1H), 3.31-3.47 (m, 2H), 7.21-7.57 (m, 5H). IR (DCM): 1447, 1714, 2956.

3-Azepan-1-yl-1-cyclopentyl-1-hydroxy-1-phenyl-propan-2-one (Compound No. 74)

Mass: (m/z): 316 (M⁺+1) ¹H NMR (CDCl₃): 1.18-1.39 (m, 23H), 2.41-2.57 (m, 4H), 3.32-3.39 (m, 2H), 7.20-7.61 (m, 5H). IR (DCM): 1447, 1712, 2934.

1-Cyclopentyl-1-hydroxy-3-(3-hydroxy-piperidin-1-yl)-1-phenyl-propan-2-one (Compound No. 76)

Mass: (m/z): 318 (M⁺+1) ¹H NMR (CDCl₃): 1.31-1.69 (m, 9H), 2.11-2.15 (m, 2H), 2.81-2.91 (m, 2H), 3.20-3.44 (m, 5H), 3.66-3.87 (m, 2H), 7.23-7.58 (m, 5H). IR (DCM): 1446, 1713, 2866, 2945, 3395.

3-(4-Benzyl-piperidin-1-yl)-1-cyclopentyl-1-hydroxy-1-phenyl-propan-2-one (Compound No. 78)

Mass: (m/z): 392.0 (M⁺+1) IR (DCM): 1736.7 cm⁻¹, 2924.1 cm⁻¹ ¹H NMR (CDCl₃):δ 1.257-1.371 (m, 8H), 1.569-1.887 (m, 8H), 2.584-2.784 (m, 4H), 3.269-3.305 (m, 2H), 5.071 (s, 1H), 7.117-7.284 (m, 10H).

(2R)-1-Cyclopentyl-1-hydroxy-3-(2-methyl-imidazol-1-yl)-1-phenyl-propan-2-one (Compound No. 81)

Mass (m/z): 299 (M⁺+1) ¹H NMR (D₂O): 1.54-1.79 (m, 8H), 2.18 (s, 3H), 3.24-3.27 (m, 1H), 5.39-5.45 (m, 2H), 7.15-7.16 (brs, 1H), 7.36-7.37 (brs, 1H), 7.51-7.60 (m, H), 7.70-7.72 (m, 2H). IR in DCM: 3399.9, 2954.8, 1728.6, 1607.9

5-(3-Cyclopentyl-3-hydroxy-2-oxo-3-phenyl-propyl)-2,5-diaza-bicyclo[2.2.1]heptane-2-carboxylic acid tert-butyl ester (Compound No. 82)

Mass (m/z): 415.0 (M⁺+1) IR (DCM): 1696.31 cm⁻¹, 2959.18 cm^{-1 1}H NMR (CDCl₃): δ 1.309-1.418 (m, 12H), 1.467-1.680 (m, 8H), 2.410-2.526 (m, 3H), 2.817-3.213 (m, 4H), 3.373-3.560 (m, 2H), 7.215-7.550 (m, 5H).

Methanesulfonic acid 3-(3-cyclopentyl-3-hydroxy-2-oxo-3-phenyl-propyl)-3-aza-bicyclo[3.2.1]oct-8-yl ester (Compound No. 83)

Mass: (m/z): 422.0 (M⁺+1) IR (DCM): 1712.16 cm⁻¹, 2949.85 cm^{-1 1}H NMR (CDCl₃): δ 1.234-1.256 (m, 2H), 1.517-1.752 (m, 14H), 2.515-2.670 (m, 4H) 2.999-3.284 (m, 2H), 4.627-4.659 (m, 1H), 7.219-7.603 (m, 5H).

(2R)-1-Cyclopentyl-1-hydroxy-3-(2-methyl-4,5-dihydro-imidazol-1-yl)-1-phenyl-propan-2-one (Compound No. 85)

Mass: (m/z): 301 (M⁺+1) ¹H NMR (D₂O, 300 MHz): 1.57-1.67 (m, 8H), 1.89 (s, 3H), 3.17-3.20 (m, 1H), 3.37 (s, 1H), 3.64-3.69 (m, 2H), 3.81-3.84 (m, 2H), 4.90-4.95 (m, 2H), 7.45-7.54 (m, 3H), 7.62-7.65 (m, 2H). IR in DCM: 3329.0, 3090.5, 2953.4, 2868.4, 1725.9, 1616.0.

### Scheme 1, Path B Procedure

### Example 12

### Preparation of 1-cyclopentyl-1-phenyl-3-(piperidin-1-yl)-propane-1,2-diol (Compound No. 13)

To a solution of 1-Cyclopentyl-1-hydroxy-1-phenyl-3-(piperidin-1-yl)-propan-2-one (0.2 g, 0.66 mM, Compound No. 14) in dry methanol (2.0 mL) at 0-5° C., sodiumborohydride (0.076 g, 2.0 mM) was added portion wise and the reaction mixture was stirred at the same temperature for 2 hour. Water (2.0 mL) was added to the reaction mixture at the same temperature and methanol was concentrated under reduced pressure. The residue was further diluted with water (8.0 ml) and the pH was adjusted to 1-2 with 1N hydrochloric acid. Washed with dichloromethane and the pH of the aqueous layer was adjusted to 13-14 with 1N sodium hydroxide. The organic compound was extracted with dichloromethane (3×25 mL) and the combined dichloromethane layer was washed with water and brine solution. Dried over anhydrous sodium sulphate and concentrated under reduced pressure to get the title organic compound.

Yield: 84% (0.168 g); m.p: 249-252°; IR (KBr): 3330.5 cm⁻¹; ¹H NMR (CDCl₃): δ 7.55-7.57 (m, 2H), 7.23-7.34 (m, 3H), 5.29-5.30 (brs, 1H, —OH), 4.39 (d, J=9 Hz, 1H), 3.67 (s, J-9 Hz, 1H), 3.67 (d, J=9 Hz, 1H), 3.39 (m, 1H), 3.08 (m, 1H), 2.94 (m, 1H), 2.56 (brs, 2H), 1.44-1.84 (m, 16H, including 2° —OH); Mass (m/z): 304 (M+1)⁺.

Analogues of 1-cyclopentyl-1-phenyl-3-piperidin-1-yl-propane-1,2-diol (Compound No. 13) described below can be prepared by reducing the appropriate ketone, as applicable in each case.

3-(3-Benzyl-3-aza-bicyclo[3.1.0]hex-6-ylmethyl)-methyl-amino]-1-cyclopentyl-1-phenyl-propane-1,2-diol (Compound No. 6)

IR (DCM): 3421.0 cm⁻¹; ¹H NMR (CDCl₃): δ 7.55-7.57 (m, 2H), 7.21-7.34 (m, 8H), 4.00-4.05 (m, 1H), 3.57 (s, 2H), 2.96 (s, 1H), 2.92-2.96 (m, 2H), 2.75 (m, 1H), 2.20-2.34 (m, 8H, including 2° —OH), 1.29-1.64 (m, 10H), 0.90 (m, 1H); Mass (m/z): 435 (M⁺+1).

1-Cyclopentyl-3-(4-methyl-piperazin-1-yl)-1-phenyl-propane-1,2-diol (Compound No. 11)

m.p: 119-124° C.; IR (DCM): 3405.7 cm⁻1; ¹H NMR (CDCl₃): δ 7.53-7.56 (m, 2H), 7.22-7.37 (m, 3H), 4.03-4.08 (m, 1H), 2.25-2.67 (m, 13H, including —OH), 1.45-1.79 (m, 11H); Mass (m/z): 319 (M⁺+1).

3-(4-Benzyl-piperazin-1-yl)-1-cyclopentyl-1-phenyl-propane-1,2-diol (Compound No. 12)

m.p: 169-175° C.; IR (KBr): 3230.3 cm⁻¹; ¹H NMR (CDCl₃): δ 7.73-7.75 (m, 2H), 7.46-7.59 (m, 8H), 4.40 (m, 1H), 3.74 (s, 2H), 2.84-2.94 (m, 8H), 2.53-2.59 (m, 2H), 1.39-2.03 (m, 10H, including 2Y—OH); Mass (m/z): 395 (M⁺+1).

1-Cyclopentyl-3-morpholin-4-yl-1-phenyl-propane-1,2-diol (Compound No. 16)

m.p: 137° C.; IR (KBr): 3378.0 cm⁻¹; ¹H NMR (CDCl₃): δ 7.54-7.57 (m, 2H), 7.29-7.48 (m, 3H), 5.08 (brs, 1H), 4.21-4.50 (m, 3H), 3.90-3.98 (m, 4H), 3.57 (m, 1H), 3.25(m, 1H), 2.96 (m, 1H), 2.82 (m, 1H), 1.45-1.66 (m, 10H, including 2° —OH); Mass (m/z): 306 (M⁺+1).

3-(1-Benzyl-piperidin-4-ylamino)-1-cyclopentyl-1-phenyl-1,2-diol (Compound No. 18)

IR (KBr): 3418.7 cm⁻¹;¹H NMR (CDCl₃): δ 7.53-7.55 (m, 2H), 7.24-7.36 (m, 8H), 3.91-3.92 (m, 1H), 3.47 (s, 2H), 2.69-2.81 (m, 6H), 2.25 (m, 2H), 1.23-2.02 (m, 12H); Mass (m/z): 409 (M⁺+1).

1-Cyclopentyl-3-(3,5-dimethyl-piperazin-1-yl)-1-phenyl-propane-1,2-diol (Compound No. 22)

Mass (m/z): 333.0 (M⁺+1) IR (DCM): 2361.4 cm^{-1 1}H NMR (CDCl₃): δ 1.053-1.094 (m, 3H), 1.203-1.256 (m, 3H), 1.583-2.021 (m, 12H), 2.279-2.645 (m, 6H), 2.975-2.985 (m, 2H), 4.057-4.104 (m, 1H), 7.222-7.555 (m, 5H). Yield: 91.45%.

1-Cyclopentyl-3-[methyl-(1-methyl-piperidin-4-yl)-amino]-1-phenyl-propane-1,2-diol (Compound No. 24) Mass (m/z): 347.0 (M⁺+1) IR (DCM): 2946.8 cm^{-1 1}H NMR (CDCl₃): δ 1.205-1.257 (m, 2H), 1.436-1.621 (m, 10H), 1.838-1.887 (m, 4H), 2.222-2.254 (m, 8H), 2.451-2.864 (m, 4H), 4.036-4.062 (m, 1H), 7.217-7.359 (m, 3H), 7.555-7.580 (m, 2H).

3-[(3-Benzyl-3-aza-bicyclo[3.1.0]hex-6-yl)-methyl-amino]-1-cyclopentyl-1-phenyl-propane-1,2-diol (Compound No. 26)

Mass (m/z): 420 (M⁺+1) 1H NMR (CDCl₃+D₂O, 300 MHz): 1.18-1.33 (m, 3H), 1.41-1.60 (m, 8H), 1.52-1.60 (m, 1H), 2.169 (brs, 1H), 2.38 (s, 3H), 2.50-2.52 (m, 2H), 2.89-3.014 (m, 2H), 3.56 (brs, 2H), 4.056-4.102 (m, 1H), 7.22-7.34 (m, 8H), 7.52-7.54 (m, 2H).

3-(1-Benzyl-pyrrolidin-3-ylamino)-1-cyclopentyl-1-propan-1,2-diol (Compound No. 29)

Mass (m/z): 395 (M⁺+1) ¹H NMR (CDCl₃): δ 1.42-1.46 (m, 7H), 1.75-1.83 (m, 1H), 2.08-2.2 (m, 1H), 2.37-2.43 (m, 2H), 2.56-2.69 (m, 8H), 3.17-3.19 (m, 1H), 3.55-3.58 (s, 2H), 3.92-3.94 (m, 1H), 7.24-7.36 (m, 8H), 7.53-7.55 (m, 2H). IR in DCM: 3417.9, 2954.3, 2866.4, 2795.9, 2513.8, 2360.7, 1656.6

1,1-Diphenyl-3-piperazin-1-yl-propane-1,2-diol (Compound No. 30)

Mass (m/z): 313.0 (M⁺+1) IR (DCM): 1596.0 cm⁻¹, 3262.9 cm^{-1 1}H NMR (CDCl₃): δ 2.21-2.29 (m, 4H), 2.37-2.40 (m, 3H), 2.52-2.64 (m, 2H), 2.91-2.94 (m, 4H), 4.63-4.67 (m, 1H), 7.17-7.43 (m, 8H), 7.59-7.62 (m, 2H).

3-[Methyl-(1-methyl-piperidin-4-yl)-amino]-1,1-diphenyl-propane-1,2-diol (Compound No. 33)

Mass (m/z): 355.0 (M++1) IR (DCM): 1637.5 cm⁻¹, 3361.0 cm^{-1 1}H NMR (CDCl₃): δ 1.52-1.71 (m, 6H), 1.90-2.13 (m, 4H), 2.27 (s, 6H), 2.65-2.92 (m, 3H), 4.56-4.60 (m, 1H), 7.16-7.42 (m, 8H), 7.61-7.63 (m, 2H).

1-Cyclopentyl-3-(3-methyl-piperazin-1-yl)-1-phenyl-propane-1,2-diol (Compound No. 40)

Mass: (m/z): 319.0 (M⁺+1) IR (DCM): 1650.8 cm⁻¹, 2956.9 cm^{-1 1}H NMR (CDCl₃): δ 1.023-1.073 (m, 4H), 1.213-1.333 (m, 6H), 1.451-1.628 (m, 6H), 1.771-1.824 (m, 4H), 2.872-2.952 (m, 4H), 4.038-4.085 (m, 1H), 7.249-7.560 (m, 5H).

1-Cyclopentyl-3-[1,4]diazepan-1-yl-1-phenyl-propane-1,2-diol (Compound No. 42)

Mass (m/z): 319.0 (M⁺+1) IR (DCM): 1493.3 cm⁻¹, 2959.3 cm^{-1 1}H NMR (CDCl₃): δ 1.257-1.335 (m, 5H), 1.417-1.555 (m, 6H), 1.942-2.009 (m, 4H), 2.575-2.845 (m, 6H), 3.127-3.493 (m, 3H), 3.951-3.994 (m, 1H), 7.236-7.552 (m, 5H).

3-[1,4′]Bipiperidinyl-1′-yl-1-cyclopentyl-1-phenyl-propane-1,2-diol (Compound No. 44)

Mass (m/z): 387 (M⁺+1) ¹H NMR (CDCl₃): 1.22-1.78 (m, 19H), 1.99-2.03 (m, 2H), 2.16-2.28 (m, 2H), 2.97-2.49 (m, 4H), 2.60-2.69 (m, 2H), 3.08-3.12 (m, 1H), 4.00-4.05 (m, 1H), 7.22-7.56 (m, 5H). IR (DCM): 1448, 1638, 2938, 3425.

1-Cyclopentyl-3-[1′-(3-cyclopentyl-2,3-dihydroxy-3-phenyl-propyl)-[4,4′]bipiperidinyl-1-yl]-1-phenyl-propane-1,2-diol (Compound No. 46)

Mass (m/z): 605 (M⁺+1) ¹H NMR (CDCl₃): 0.90-0.98 (m, 4H), 1.03-1.98 (m, 34H), 2.64-2.69 (m, 2H), 2.98-3.01 (m, 2H), 7.16-7.52 (m, 10). IR (DCM): 1646, 2947, 3443.

3-(1-Benzyl-pyrrolidin-3-ylamino)-1,1-diphenyl-propane-1,2-diol (Compound No. 47)

Mass (m/z): 403 (M⁺+1) ¹H NMR (CDCl₃): 1.96-2.00 (m, 2H), 2.25-2.60 (m, 6H), 2.75-2.81 (m, 2H), 3.05-3.10 (m, 2H), 3.55 (s, 2H), 4.55 (s, 1H), 7.17-7.60 (m, 15H). IR (DCM): 1449, 1656, 2795, 2960, 3445.

3-[4-(2-Benzo[1,3]dioxol-5-yl-ethyl)-[1,4]diazepan-1-yl]-1-cyclopentyl-1-phenyl-propane-1,2-diol (Compound No. 51)

Mass (m/z): 467.0 (M⁺+1) IR (DCM): 1568.8 cm^{—1}, 3408.4 cm^{-1 1}H NMR (CDCl₃): δ 1.17-1.25 (m, 4H), 1.46-1.60 (m, 4H), 1.83-2.04 (m, 4H), 2.39-2.45 (m, 4H), 2.59-2.85 (m, 11H), 4.06 (s, 1H), 5.92 (s, 2H), 6.60-6.74 (m, 3H), 7.31-7.57 (m, 5H).

1-Cyclopentyl-3-dimethylamino-1-phenyl-propane-1,2-diol (Compound No. 54)

Mass (m/z): 264.0 (M⁺+1) IR (DCM): 1574.8 cm⁻¹, 2953.3 cm^{-1 1}H NMR (CDCl₃): δ 1.166-1.254 (m, 4H), 1.459-1.609 (m, 4H), 1.997-2.043 (m, 2H), 2.298-2.461 (m, 8H), 2.734-2.761 (m, 1H), 4.046-4.091 (m, 1H), 7.221-7.571 (m, 5H).

3-[1,4]Diazepan-1-yl-1,1-diphenyl-propane-1,2-diol (Compound No. 56)

Mass (m/z): 327 (M⁺+1) ¹H NMR (CDCl₃, 300 MHz): 1.84-1.88 (m, 2H), 2.32-2.33 (m, 1H), 2.56-2.74 (m, 3H), 2.75-2.82 (m, 3H), 2.98-3.106 (m, 3H), 3.42-3.64 (brs, 3H), 4.56-4.60 (m, 1H), 7.17-7.40 (m, 8H), 7.59-7.62 (m, 2H). IR in DCM: 3416.6, 3057.7, 2932.5, 2845.8, 1600.1

3-[4-(2-Benzo[1,3]dioxol-5-yl-ethyl)-[1,4]diazepan-1-yl]-1,1-diphenyl-propane-1,2-diol (Compound No. 58)

Mass (m/z): 475 (M⁺+1) ¹H NMR (CDCl₃, 300 MHz): 1.91 (brs, 2H), 2.37-2.41 (m, 2H), 2.66-2.85 (m, 12H), 4.66-4.68 (m, 1H), 5.92 (s, 2H), 6.61-6.74 (m, 2H), 7.19-7.43 (m, 8H), 7.59-7.62 (m, 2H). IR in DCM: 3396.5, 3014.4, 2920.3, 1657.7

1-Cyclopentyl-3-(4-hydroxy-piperidin-1-yl)-1-phenyl-propane-1,2-diol (Compound No. 59) IR in DCM: 3405.4, 2950.6, 2867.2, 1637.9 Mass (m/z): 320 (M⁺+1) ¹H NMR (CDCl₃, 300 MHz): 1.43-1.61 (m, 10H), 1.72-1.88 (m, 5H), 2.18-2.21 (m, 3H), 2.29-2.31 (m, 2H), 2.63-2.69 (m, 2H), 2.82 (brs, 1H), 3.65-3.73 (m, 1H), 3.99-4.03 (m, 1H), 7.27-7.35 (m, 3H), 7.54-7.56 (m, 2H).

1-Cyclopentyl-3-(2-dimethylamino-ethylamino)-1-phenyl-propane-1,2-diol (Compound No. 60)

Mass (m/z): 307.0 (M⁺+1) IR (DCM): 2953.5 cm⁻¹, 3385.4 cm^{-1 1}H NMR (CDCl₃): δ 1.417-1.458 (m, 4H), 1.540-1.558 (m, 4H), 2.201-2.309 (m, 10H), 2.371-2.410 (m, 2H), 2.642-2.706 (m, 4H), 3.971-3.991 (m, 1H), 7.219-7.360 (m, 3H), 7.536-7.561 (m, 2H).

1-Cyclopentyl-3-(4-methyl-[1,4]diazepan-1-yl)-1-phenyl-propane-1,2-diol (Compound No. 62)

Mass (m/z): 333.0 (M⁺+1) IR (DCM): 1638.5 cm⁻¹, 3420.8 cm^{-1 1}H NMR (CDCl₃): δ 1.21-1.25 (m, 2H), 1.45-1.62 (m, 6H), 1.79-1.86 (m, 3H), 2.35-2.44 (m, 7H), 2.65-2.76 (m, 8H), 3.96-4.01 (m, 1H), 7.22-7.57 (m, 5H).

1-[1,4]Diazepan-1-yl-4-methyl-3-phenyl-pentane-2,3-diol (Compound No. 64)

Mass (m/z): 293 (M⁺+1) ¹H NMR (CDCl₃, 300 MHz): 0.69-0.71 (m, 3H), 0.94-0.96 (m, 3H), 1.86-1.90 (m, 2H), 2.24-2.28 (m, 1H), 2.46-2.55 (m, 2H), 2.69-2.77 (m, 4H), 3.05 (brs, 4H), 3.48-3.61 (brs, 3H), 4.13-4.18 (m, 1H), 7.24-7.35 (m, 3H), 7.50-7.53 (m, 2H). IR in DCM: 3854.2, 3411.6, 2931.9, 1645.3, 1576.8

1-Cyclopentyl-3-imidazol-1-yl-1-phenyl-propane-1,2-diol (Compound No. 66)

Mass (m/z): 287.0 (M⁺+1) IR (DCM): 2960.8 cm⁻¹, 3382.9 cm^{-1 1}H NMR (CDCl₃): δ 1.25-1.64 (m, 9H), 2.87-2.96 (m, 2H), 3.27-3.32 (m, 1H), 3.93-3.97 (m, 1H), 4.19-4.24 (m, 1H), 6.84-7.59 (m, 8H).

1-Cyclopentyl-3-(2-methyl-imidazol-1-yl)-1-phenyl-propane-1,2-diol (Compound No. 68)

Mass (m/z): 301.0 (M⁺+1) IR (DCM): 1719.3 cm⁻¹, 2958.3 cm^{-1 1}H NMR (CDCl₃): δ 1.31-1.62 (m, 6H), 2.01-2.04 (m, 2H), 2.22 (s, 3H), 2.89-3.32 (m, 2H), 3.95-4.12 (m, 4H), 6.71-7.61 (m, 7H).

1-Cyclopentyl-3-(2-isopropyl-imidazol-1-yl)-1-phenyl-propane-1,2-diol (Compound No. 70)

Mass (m/z): 329.0 (M⁺+1) IR (DCM): 2962.1 cm⁻¹, 3395.3 cm^{-1 1}H NMR (CDCl₃): δ 1.11-1.25 (m, 8H), 1.46-1.62 (m, 5H), 2.02-2.04 (m, 2H), 2.83-2.89 (m, 2H), 3.29-3.37 (m, 1H), 3.95-3.98 (m, 2H), 4.16-4.21 (m, 1H), 6.81-7.59 (m, 7H). m.p: 117.0°-118.0° C.

1-Cyclopentyl-1-phenyl-3-pyrrolidin-1-yl-propane-1,2-diol (Compound No. 73)

Mass (m/z): 290 (M⁺+1) ¹H NMR (CDCl₃): 1.18-1.80 (m, 15H), 2.41-2.49 (m, 2H), 2.61-2.72 (m, 4H) 4.02-4.06 (m, 1H), 7.22-7.57 (m, 5H). IR (DCM): 1447, 2814, 2953, 3382.

3-Azepan-1-yl-1-cyclopentyl-1-phenyl-propane-1,2-diol (Compound No. 75)

Mass (m/z): 318 (M⁺+1) ¹H NMR (CDCl₃): 1.19-1.95 (m, 16H), 2.38-2.42 (m, 1H), 2.90-3.07 (m, 6H), 4.25-4.27 (m, 1H), 7.22-7.58 (m, 5H). IR (DCM): 1064, 1446, 2939, 3359.

1-Cyclopentyl-3-(3-hydroxy-piperidin-1-yl)-1-phenyl-propane-1,2-diol (Compound No. 77)

Mass (m/z): 320 (M⁺+1) ¹H NMR (CDCl₃): 1.17-1.25 (m, 4H), 1.125-1.52 (m, 8H), 1.73-1.81 (m, 4H), 2.24-2.32 (m, 3H), 3.81-3.83 (m, 1H), 4.04-4.09 (m, 1H), 7.22-7.55 (m, 5H). IR (DCM): 1061, 1444, 2945, 3384.

1-Cyclopentyl-3-(2-dimethylamino-ethylamino)-1-phenyl-propane-1,2-diol (Compound No. 80)

Mass (m/z): 307 (M⁺+1) ¹H NMR (CDCl₃, 300 MHz): 1.20-1.25 (brs, 2H), 1.42-1.56 (m, 6H), 1.80 (brs, 1H) 2.11-2.19 (brs, 6H), 2.31-2.39 (m, 2H), 2.62-2.69 (m, 4H), 3.01 (brs, 3H), 3.97-3.99 (m, 1H), 7.22-7.36 (m, 3H), 7.53-7.56 (m, 2H). IR in DCM: 3384.5, 2948.8, 2863.8, 2780.7, 1718.6, 1596.1

### Example 13

### Preparation of 1-cyclopentyl-1-phenyl-3(-piperazin-1-yl)-propane-1,2-diol (Compound No. 10)

To a solution of 3-(4-Benzyl-piperazin-1-yl)-1-cyclopentyl-1-phenyl-propane-1,2-diol (0.2 g, 0.63 mM) in a mixture of methanol (12.5 mL) and tetrahydrofuran (5.0 mL) was added palladium or carbon (0.1 g, 10%) followed by the addition of ammonium formate (0.2 g) and the reaction mixture was refluxed for 30 minutes. The reaction mixture was cooled and filtered through celite pad and the bed was washed with methanol (10.0 mL), ethyl acetate (10.0 mL) and water (10.0 mL). The filtrate was concentrated under reduced pressure to remove the organic solvent. The residue thus obtained was diluted with water (10.0 mL) and extracted with dichloromethane (3×25.0 mL). The combined dichloromethane layer was washed with water and brine solution, dried over anhydrous sodium sulphate and concentrated under reduced pressure to get the title organic compound. Yield: 77% (0.15 g); IR (DCM): 3414.9 cm⁻¹; ¹H NMR (CDCl₃): δ 7.53-7.56 (m, 2H), 7.25-7.46 (m, 3H), 4.04-4.08 (m, 1H), 2.20-2.94 (m, 11H, including 3° —OH), 1.21-1.62 (m, 11H, including 2° —OH and —NH); Mass (m/z): 306 (M⁺+1).

Analogues of 1-cyclopentyl-1-phenyl-3-piperazin-1-yl-propane-1,2-diol (Compound No. 10) described below can be prepared by debenzylating the appropriate amine, as applicable in each case.

3-(3-aza-bicyclo[3.1.0]hex-6-ylamino)-1-cyclopentyl-1-phenyl-propane-1,2-diol (Compound No. 3)

The title compound was synthesised by following the usual N-debenzylation.

m.p: 50° C.; IR (DCM): ¹H NMR (CDCl₃): δ 7.51-7.56 (m, 2H), 7.16-7.37 (m, 3H), 3.94-3.98 (m, 1H), 2.75-3.00 (m, 4H), 2.54-2.60 (m, 1H), 1.80-1.85 (m, 2H), 1.18-1.5 (m, 11H); Mass (m/z): 317 (M⁺+1).

3-(3-aza-bicyclo[3.1.0]hex-6-ylamino)-1,1-diphenyl-propane-1,2-diol (Compound No. 5)

m.p: 85° C. (softening starts); IR (KBr): 3319.4 cm⁻¹; ¹H NMR (CDCl₃+CD₃OD): δ 7.21-7.60 (m, 10H), 4.61-4.62 (m, 1H), 3.36 (s, 1H, —OH), 2.64-2.99 (m, 6H), 1.83 (s, 1H, —OH), 1.27-1.54 (m, 5H, including —NH); Mass (m/z): 325 (M⁺+1).

3-[(3-Aza-bicyclo[3.1.0]hex-6-ylmethyl)-methyl-amino]-1-cyclopentyl-1-phenyl-propane-1,2-diol (Compound No. 9) IR (DCM): 3407.9 cm⁻¹; Mass (m/z): 345 (M⁺+1).

### Scheme I, Path b2 Procedure

### Example 13a

### 3-(4-Methyl-piperazin-1-yl)-1,1-diphenyl-propane-1,2-diol (Compound No. 31)

To a solution of Compound No. 30 (0.4 g, 1.22 mmol) in acetonitrile (20.0 ml) and formaldehyde (37%, 2.6 ml), was added sodium cyanoborohydride (0.26 g, 4.2 mmol) at 25-30° C. The reaction mixture was stirred for 1 hour and subsequently neutralized with acetic acid (1.8 ml). The reaction mixture was again stirred for 12 hours at the same temperature. The solvent was removed under reduced pressure and the residue thus obtained was diluted with water and basified to pH=14 with sodium hydroxide (10%). The reaction mixture was extracted with ethylacetate, dried over anhydrous sodium sulphate and concentrated under reduced pressure. The residue thus obtained was purified by column chromatography using ethyl acetate in hexane as eluent to furnish the title compound. Yield: 66.26%.

Mass (m/z): 327.0 (M⁺+1) ¹H NMR (CDCl₃, 300 MHz): 2939.1 cm^{-1 NMR (CDCl}₃): 2.00-2.24 (m, 10H), 2.31-2.62 (m, 5H), 4.64-4.68 (m, 1H), 7.17-7.44 (m, 8H), 7.59-7.62 (m, 2H).

### Scheme I, Path A Procedure

### Example 14

### Preparation of 3-[(3-benzyl-3-aza-bicyclo[3.1.0]hex-6-ylmethyl)-methyl-amino]-1-cyclopentyl-1-hydroxy-1-phenyl-propan-2-one-oxime (Compound No. 8)

### Step a: Preparation of 3-[(3-benzyl-3-azabicyclo[3.1.0]hex-6-ylmethyl)-methyl-amino]-1-cyclopentyl-1-hydroxy-1-phenyl-propan-2-one

To a solution of a compound (1α, 5α, 6α)-6-methylamino-methyl-3-benzyl-3-azabicyclo[3.1.0]hexane (0.36 g, 1.68 mL) in triethylamine (0.47 m, 3.36 mM) was added N,N-dimethylamino pyridine (0.02 gm), dichloromethane (10 mL) and 3-bromo-1-cyclopentyl-1-hydroxy-1-phenyl-2-propanone (0.5 gm, 1.68 mM). The resulting reaction mixture was stirred at room temperature for 1 hour followed by the addition of saturated solution of sodium bicarbonate solution (5.0 mL). The reaction mixture was stirred for five minutes. The organic layer was separated, washed with water and brine solution. The organic layer was dried over anhydrous sodium sulphate and concentrated under reduced pressure. The residue thus obtained was purified with column chromatography using 50% ethyl acetate in hexane solvent mixture as an eluent.

Yield: 68% (0.5 g);

### Step b: Preparation of 3-[(3-benzyl-3-aza-bicyclo[3.1.0]hex-6-ylmethyl)-methyl-amino]-1-cyclopentyl-1-hydroxy-1-phenyl-propan-2-one-oxime (Compound No. 8)

To a solution of a compound obtained from step a above (0.5 g, 1.1 mM, step a) in ethanol (10.0 mL) was added hydroxylamine hydrochloride (1.0 g, 14.4 mM) and pyridine (1.3 ml, 16.0 mM). The resulting reaction mixture was refluxed for 30 hours followed by cooling to room temperature. Ethanol was evaporated off under reduced pressure. The residue was diluted with water and extracted with ethyl acetate (3×25 mL). The ethyl acetate layer was washed with water and brine solution, dried over anhydrous sodium sulphate and concentrated under reduced pressure. The residue was purified by silica gel column chromatography using 70% ethyl acetate in hexane to get the title compound.

Yield: 23% (0.12 g); IR (DCM): 3419.1, 1652.2 cm⁻¹; Mass (m/z): 448 (M⁺+1).

Analogous of 3-[(3-benzyl-3-aza-bicyclo[3.1.0]hex-6-ylmethyl)-methyl-amino]-1-cyclopentyl-1-hydroxy-1-phenyl-propan-2-one-oxime (Compound No. 8) described below was prepared similarly,

3-(1-Benzyl-piperidin-4-ylamino)-1-cyclopentyl-1-hydroxy-1-phenyl-propan-2-one oxime (Compound No. 20)

Mass (m/z): 421.0 (M⁺+1) IR (DCM): 1729.4 cm⁻¹, 3479.2 cm^{-1 1}H NMR (CDCl₃): δ 1.42-1.60 (m, 4H), 1.89-2.10 (m, 4H), 2.36-2.60 (m, 8H), 2.85-3.29 (m, 5H), 3.39-3.80 (m, 4H), 7.21-7.47 (m, 8H), 7.66-7.72 (m, 2H).

### Scheme 1, Path b1 Procedure

### Example 14a

### 3-[4-(4-Chloro-benzyl)-piperazin-1-yl]-1-cyclopentyl-1-phenyl-propane-1,2-diol (Compound No. 36)

To a solution of Compound No. 30 (0.3 g, 0.9 mmol) and 1-bromomethyl-4-chloro-benzene (0.23 g, 1.0 mmol) in acetonitrile (10.0 ml), was added potassium carbonate (0.38 g, 2.7 mmol) and potassium iodide (0.3 g, 1.8 mmol). The reaction mixture was refluxed for 12 hours. The solvent was concentrated under reduced pressure and the residue thus obtained was diluted with ethyl acetate and water. The ethyl acetate layer was concentrated under reduced pressure. The residue thus obtained was purified by column chromatography using ethyl acetate in hexane as eluent to furnish the title compound.

Yield: 87.45% Mass: (m/z): 429.44 (M⁺+1) IR (DCM): 1639.9 cm⁻¹, 3441.3 cm^{-1 1}H NMR (CDCl₃): δ 1.172-1.322 (m, 8H), 1.476-1.797 (m, 6H), 2.069-2.668 (m, 6H), 3.462-4.075 (m, 4H), 7.209-7.354 (m, 7H), 7.526-7.551 (m, 2H).

### Scheme 1, Path C Procedure

### Example 14b

### 3-[(1-Benzyl-piperidin-4-yl)-methyl-amino]-1-cyclopentyl-1-hydroxy-1-phenyl-propan-2-one (Compound No. 19

To a solution of Compound No. 28 (0.495 g, 1.22 mmol) in acetonitrile (20.0 ml) and formaldehyde (37%, 2.6 ml), was added sodium cyanoborohydride (0.26 g, 4.2 mmol) at 25-30° C. The reaction mixture was stirred for 1 hour and subsequently neutralized with acetic acid (1.8 ml). The reaction mixture was again stirred for 12 hours at the same temperature. The solvent was removed under reduced pressure and the residue thus obtained was diluted with water and basified to pH=14 with sodium hydroxide (10%). The reaction mixture was extracted with ethylacetate, dried over anhydrous sodium sulphate and concentrated under reduced pressure. The residue thus obtained was purified by column chromatography using ethyl acetate in hexane as eluent to furnish the title compound. Yield: 0.132 g

Mass: (m/z): 421.0 (M⁺+1) IR(DCM): 1715.0 cm⁻¹, 2856.3 cm^{-1 1}H NMR (CDCl₃): δ 1.274-1.692 (m, 14H), 1.781-1.871 (m, 3H), 2.056-2.301 (m, 2H), 2.839-2.893 (m, 3H), 3.317-3.476 (m, 4H), 7.244-7.354 (m, 8H), 7.571-7.597 (m, 2H).

Following compound was prepared similarly,

3-[(1-Benzyl-pyrrolidin-3-yl)-methyl-amino]-1-cyclopentyl-1-hydroxy-1-phenyl-propan-2-one (Compound No. 35)

Mass (m/z): 407 (M⁺+1) ¹H NMR (CDCl₃, 300 MHz): δ 1.28-1.32 (m, 2H), 1.48-1.71 (m, 8H), 1.90-2.04 (m, 4H), 2.55-2.72 (m, 4H), 2.85-2.95 (m, 1H), 3.25-3.38 (m, 3H), 3.61-3.66 (m, 2H), 7.21-7.32 (m, 8H), 7.53-7.56 (m, 2H). IR in DCM: 3447.3, 2956.3, 2792.4, 1713.8

### Scheme 1, Path d Procedure

### Example 14c

### Methanesulfonic acid 3-(3-cyclopentyl-3-hydroxy-2-oxo-3-phenyl-propyl)-3-aza-bicyclo[3.2.1]oct-8-yl ester (Compound No. 84)

The title compound was prepared following the procedure as described in Example 13, by using Compound No. 82.

Mass (m/z): 315.0 (M⁺+1) IR (DCM): 1724.8 cm⁻¹, 2956.8 cm⁻¹, 3385.0 cm^{-1 1}H NMR (CDCl₃, 300 MHz): δ 1.220-1.704 (m, 8H), 2.275-2.511 (m, 2H), 3.160-3.460 (m, 2H), 3.582-3.726 (m, 4H), 4.509 (s, 1H), 4.654-4.741 (m, 4H), 7.500-7.678 (m, 5H).

### Scheme 2, Path a Procedure

### Example 15

### Preparation of (R or S) 3-(3-benzyl-3-aza-bicyclo[3.1.0]hex-6-ylamino)-1-cyclopentyl-1-phenyl-propane-1,2-diol (Compound No. 1)

To a solution of polar epoxide-B of 1-cyclopentyl-1-hydroxy-1-phenyl 2,3-epoxy-propane (0.45 g, 2.0 mM) in dry ethanol (15.0 mL) was added (1α, 5α, 6α)-6-amino-3-benzyl-3-azabicyclo[3.1.0]hexane (synthesis reported in T. F. Braish et. al. synlett 1996, 1100) (0.35 g, 1.9 mM) and the reaction mixture was refluxed for 24 hours. Ethanol was evaporated under reduced pressure. The residue was purified through column chromatography using 5% methanol in dichloromethane solvent mixture as an eluent to get the title organic compound.

Yield: 40% (0.3 g); m.p: 138-144° C.; IR (KBr): 3464.5 cm⁻¹; ¹H NMR (CDCl₃): δ 7.50-7.53 (m, 2H), 7.2-7.35 (m, 8H), 3.93-3.97 (m, 1H), 3.53 (s, 2H), 2.78-2.95 (m, 2HO, 2.76-2.78 (m, 2H), 2.33-2.60 (m, 3H), 1.80 (m, 1H), 1.26-1.53 (m, 10H); Mass (m/z): 407 (M⁺+1).

Analogues of (R or S) 3-(3-benzyl-3-aza-bicyclo[3.1.0]hex-6-ylamino)-1-cyclopentyl-1-phenyl-propane-1,2-diol (Compound No. 1) described below can be prepared by replacing appropriate amine in place of in place of (1α, 5α, 6α)-6-amino-3-benzyl-3-azabicyclo[3.1.0]hexane.

(R or S) 3-(3-Benzyl-3-aza-bicyclo[3.1.0]hex-6-ylamino)-1,1-diphenyl-propane-1,2-diol (Compound No. 2)

m.p.: 156-160° C.; IR (KBr): 3402.2 cm⁻¹; ¹H NMR (CDCl₃): δ 7.57-7.59 (m, 2H), 7.44-7.46 (m, 2H), 7.16-7.34 (m, 11H), 4.56-4.58 (m, 1H), 3.50 (s, 2H), 2.68-2.92 (m, 4H), 2.26-2.35 (m, 2H), 1.16-1.41 (m, 3H); Mass (m/z): 415 (M⁺+1).

### Example 16

### Preparation of (R or S) 3-[(3-Benzyl-3-aza-bicyclo[3.1.0]hex-6-ylmethyl-amino]-1-cyclopentyl-1-phenyl-propane-1,2-diol (Compound No. 4)

The title compound was prepared by following the procedure mentioned for the synthesis of Compound No. 11 by using the polar epoxide-B of 1-cyclopentyl-1-hydroxy-1-phenyl-2,3-epoxy propane and (1α, 5α, 6α)-6-aminomethyl-3-benzyl-3-azabicyclo[3.1.0]hexane (synthesis reported in EP 0 413 455) in place of (1α, 5α, 6α)-6-amino-3-benzyl-3-aza-bicyclo[3.1.0]hexane.

IR (DCM): 3384.4 cm⁻¹; ¹H NMR (CDCl₃): δ 7.52-7.54 (m, 2H), 7.21-7.32 (m, 8H), 4.24-4.28 (m, 1H), 3.57 (s, 2H), 2.94-3.07 (m, 4H), 2.58-2.66 (m, 4H), 1.90 (m, 1H), 1.19-1.53 (m, 8H), 0.88-1.00 (m, 3H); Mass (m/z): 421 (M⁺+1).

### Example 17

### Preparation of (R or S) 3-(3-benzyl-3-aza-bicyclo[3.1.0]hex-6-ylamino)-1-cyclopentyl-1-phenyl-propane-1,2-diol (Compound No. 7)

The title compound was prepared by following the procedure mentioned for the synthesis of Compound No. 1, using the non-polar epoxide-A of 1-cyclopentyl-1-hydroxy-1-phenyl-2,3-epoxy propane in place of polar epoxide-B of 1-cyclopentyl-1-hydroxy-1-phenyl-2,3-epoxy propane.

IR (DCM): 3442.1 cm⁻¹; ¹H NMR(CDCl₃): δ 7.18-7.38 (m, 10H), 3.98-4.01 (m, 1H), 3.48-3.49 (m, 2H), 2.77-2.90 (m, 2H), 2.17-2.60 (m, 5H, including as of the —OH), 1.90 (m, 1H), 1.06-1.46 (m, 11H); Mass (m/z): 407 (M⁺+1).

### Scheme V Procedure

### Example 18

### 1-Cyclopentyl-3-{4-[2-(2,3-dihydro-benzofuran-5-yl)-ethyl]-[1,4]diazepan-1-yl}-1-hydroxy-1-phenyl-propan-2-one (Compound No. 50)

To a solution of Compound No. 39 (0.28 g, 0.9 mmol) and 1-bromomethyl-4-chloro-benzene (0.23 g, 1.0 mmol) in acetonitrile (10.0 ml), was added potassium carbonate (0.38 g, 2.7 mmol) and potassium iodide (0.3 g, 1.8 mmol). The reaction mixture was refluxed for 12 hours. The solvent was concentrated under reduced pressure and the residue thus obtained was diluted with ethyl acetate and water. The ethyl acetate layer was concentrated under reduced pressure. The residue thus obtained was purified by column chromatography using ethyl acetate in hexane as eluent to furnish the title compound.

Yield: 59.78% Mass (m/z): 463.0 (M⁺+1) IR (DCM): 1714.6 cm⁻¹, 3444.0 cm^{-1 1}H NMR (CDCl₃, 300 MHz): δ 1.26-1.34 (m, 2H), 1.47-1.57 (m, 4H), 1.78-2.04 (m, 4H), 2.55-2.66 (m, 4H), 2.74-2.90 (m, 6H), 3.14-3.20 (m, 4H), 3.36-3.46 (m, 4H), 4.51-4.57 (m, 2H), 6.68-7.00 (m, 3H), 7.31-7.57 (m, 5H).

Following compound was prepared similarly,

3-[4-(2-Benzo[1,3]dioxol-5-yl-ethyl)-[1,4]diazepan-1-yl]-1-cyclopentyl-1-phenyl-1-propane-1,2-diol (Compound No. 49)

Mass (m/z): 465.0 (M⁺+1) IR (DCM): 1712.0 cm⁻¹, 3452.0 cm^{-1 1}H NMR (CDCl₃, 300 MHz): δ 1.25-1.34 (m, 4H), 1.37-1.69 (m, 8H), 2.47-2.53 (m, 10H), 2.59-2.73 (m, 2H), 3.30-3.46 (m, 2H), 5.91 (s, 2H), 6.59-6.73 (m, 3H), 7.21-7.59 (m, 5H).

### Example 19

### 1-Cyclopentyl-3-{4-[2-(2,3-dihydro-benzofuran-5-yl)-ethyl]-[1,4]diazepan-1-yl}-1-phenyl-propane-1,2-diol (Compound No. 52)

The title compound was prepared using the procedure as described in Example 10, by reducing Compound No. 50.

Mass (m/z) 465.0 (M⁺+1) IR (DCM): 1562.4 cm⁻¹, 3424.2 cm^{-1 1}H NMR (CDCl₃): δ 1.160-1.585 (m, 6H), 1.927-2.039 (m, 4H), 2.595-2.65 (m, 12H), 2.737-2.890 (m, 5H), 3.143-3.199 (m, 2H), 4.068 (s, 1H), 4.512-4.570 (m, 2H), 6.680-7.007 (m, 3H), 7.262-7.566 (m, 5H).

Following compounds were prepared similarly,

3-[4-(2-Benzo[1,3]dioxol-5-yl-ethyl)-piperazin-1-yl]-1-cyclopentyl-1-phenyl-propane-1,2-diol (Compound No. 37)

Mass (m/z): 453.0 (M⁺+1) IR (DCM): 1639.3 cm⁻¹, 2960.8 cm^{-1 1}H NMR (CDCl₃): δ 1.11-1.37 (m, 10H), 2.01-2.68 (m, 8H), 3.38-3.91 (m, 8H), 5.92 (s, 2H), 6.68-6.72 (m, 3H), 7.32-7.56 (m, 5H).

3-[4-(2,3-dihydro-Benzofuran-5-yl-ethyl)-piperazin-1-yl]-1-cyclopentyl-1-phenyl-propane-1,2-diol (Compound No. 38)

Mass (m/z): 451.0 (M⁺+1) IR (DCM): 1639.6 cm⁻¹, 2923.0 cm^{-1 1}H NMR (CDCl₃): δ 1.109-1.481 (m, 12H), 1.632-1.724 (m, 2H), 3.401-3.437 (m, 8H), 3.546-3.665 (m, 6H), 4.516-4.573 (m, 2H), 6.86-7.019 (m, 4H), 7.318-7.560 (m, 4H).

1-Cyclopentyl-3-[4-(3-methyl-but-2-enyl)-piperazin-1-yl]-1-phenyl-propane-1,2-diol (Compound No. 48)

Mass (m/z): 373.0 (M⁺+1) IR (DCM): 1638.3 cm⁻¹, 2959.2 cm^{-1 1}H NMR (CDCl₃): δ 1.187-1.333 (m, 11H), 1.450-1.570 (m, 5H), 1.642-1.732 (m, 6H), 2.308-2.367 (m, 2H), 2.440-2.676 (m, 6H), 4.018-4.065 (m, 1H), 7.243-7.561 (m, 5H).

3-[4-(2-Benzo[1,3]dioxol-5-yl-ethyl)-[1,4]diazepan-1-yl]-1-cyclopentyl-1-phenyl-propane-1,2-diol (Compound No. 79)

Mass (m/z): 473.0 (M⁺+1) IR (DCM): 1640.3 cm⁻¹, 2922.7 cm⁻¹ NMR (CDCl₃): 1.660-1.702 (m, 2H), 2.531-2.666 (m, 4H), 3.389-3.532 (m, 5H), 4.239-4.244 (m, 2H), 4.569-4.588 (m, 4H), 7.235-7.441 (m, 15H).

### Example 20

### 3-[(3-Aza-bicyclo[3.1.0]hex-6-yl)-methyl-amino]-1-cyclopentyl-1-hydroxy-1-phenyl-propan-2-one (Compound No. 27)

The title compound was prepared following the procedure as describe for the synthesis of Compound No. 10 by using Compound No. 25.

Mass (m/z): 329 (M⁺+1) ¹H NMR (CDCl₃, 300 MHz): 1.35-1.38 (m, 4H), 1.41-1.57 (m, 4H), 1.66-1.67 (brs, 1H), 1.79 (s, 1H), 1.96 (s, 1H), 2.095 (s, 3H), 2.14-2.22 (brs, 1H), 2.59 (brs, 2H), 2.88-3.03 (m, 4H), 3.413-3.418 (m, 2H), 7.20-7.37 (m, 3H), 7.51-7.53 (m, 2H).

### Example 21

### Biological Activity—Radioligand Binding Assays

The affinity of test compounds for M₂ and M₃ muscarinic receptor subtypes was determined by [³H]-N-methylscopolamine binding studies using rat heart and submandibular gland respectively as described by Moriya et al., (Life Sci, 1999,64(25):2351-2358) with minor modifications.

Membrane preparation: Rat submandibular glands and heart were isolated and placed in ice cold homogenising buffer (HEPES 20 mM, 10 mM EDTA, pH 7.4) immediately after sacrifice. The tissues were homogenised in 10 volumes of homogenising buffer and the homogenate was filtered through two layers of wet gauze and filtrate was centrifuged at 500 g for 10 min at −4° C. The supernatant was subsequently centrifuged at 40,000 g for 20 min at −4° C. The pellet thus obtained was resuspended in assay buffer (HEPES 20 mM, EDTA 5 mM, pH 7.4) and were stored at −70° C. until the time of assay.

Ligand binding assay: The compounds were dissolved and diluted in DMSO. The membrane homogenates (150-250 μg protein) were incubated in 250 μl of assay volume (HEPES 20 mM, pH 7.4) at 24-25° C. for 3 h. Non-specific binding was determined in the presence of 1 μM atropine. The incubation was terminated by vacuum filtration over GF/B fiber filters (Wallac). The filters were then washed with ice cold 50 mM Tris HCl buffer (pH 7.4). The filter mats were dried and bound radioactivity retained on filters was counted. The IC₅₀ & Kd were estimated by using the non-linear curve fitting program using G Pad Prism software. The value of inhibition constant Ki was calculated from competitive binding studies by using Cheng & Prusoff equation (Biochem Pharmacol, 1973,22: 3099-3108), Kᵢ=IC₅₀ /(1+L/K_{d}), where L is the concentration of [³H]NMS used in the particular experiment. pKᵢ is −log [Kᵢ].

### Example 19

### Functional Experiments Using Isolated Rat Bladder

### Methodology:

Animals were euthanized by overdose of thiopentone and whole bladder was isolated and removed rapidly and placed in ice cold Tyrode buffer with the following composition (mMol/L) NaCl 137; KCl 2.7; CaCl₂ 1.8; MgCl₂ 0.1; NaHCO₃ 11.9; NaH₂PO₄ 0.4; Glucose 5.55 and continuously gassed with 95% O₂ and 5% CO₂.

The bladder was cut into longitudinal strips (3 mm wide and 5-6 mm long) and mounted in 10 ml organ baths at 30° C., with one end connected to the base of the tissue holder and the other end connected through a force displacement transducer. Each tissue was maintained at a constant basal tension of 1 g and allowed to equilibrate for 1.5 hours during which the Tyrode buffer was changed every 15-20 min. At the end of equilibration period the stabilization of the tissue contractile response was assessed with 1 μmol/L of Carbachol until a reproducible response was obtained. Subsequently a cumulative concentration response curve to carbachol (10⁻⁹ mol/L to 3×10⁻⁴ mol/L) was obtained. After several washes, once the baseline was achieved, cumulative concentration response curves were obtained in the presence of NCE (NCE added 20 min. prior to the second cumulative response curve).

The contractile results were expressed as % of control E max. ED₅₀ values were calculated by fitting a non-linear regression curve (Graph Pad Prism). pK_{b} values were calculated by the formula pK_{b}=−log [(molar concentration of antagonist/(dose ratio−1))]where, the dose ratio=ED₅₀ in the presence of antagonist/ED₅₀ in the absence of antagonist.

In Vivo Experiments using Anesthetized Rabbit:

In vivo experiments using anesthetized rabbit Methodology Male rabbits were anesthetized with urethane 1.5 g/kg intravenously. Trachea was cannulated to maintain the patency of airway. Femoral vein and femoral arteries of both sides were cannulated for the administration of vehicle or drug substances for the measurement of BP and administration of carbachol intra-arterially respectively.

Polyethylene tubing was introduced into the bladder through the urethra and tied at the neck of the bladder. The other end of the catheter was connected to the Grass polygraph and power lab system through a Statham pressure transducer. The bladder was filled with warm (37° C.) saline.

Both the ureters were catherised to drain the urine coming from kidneys.

A stabilization period of 30-60 min. was allowed for stabilization of parameters from surgical procedures.

Salivary response was assessed by measuring the weight of a preweighted cotton gauze kept for 2 minutes in the buccal cavity immediately after the carbachol challenge.

At the end of stabilization period 2 control responses to carbachol (1.5, ug/kg intra-arterial) on bladder pressure and salivation were obtained and this response was considered as 100%. Subsequently, the effect of increasing dose of NCE was examined. The change in bladder pressure and salivation were expressed as % change from pretreatment control averages. The ID₅₀values for salivation and bladder pressure inhibition were calculated using Graph Pad Prism software, by fitting the values at dose into non-linear regression curve. Oxybutynin and Tolterodine were used as standards for comparison.

The bladder selectivity to salivation was calculated by using following formula and expressed as fold of selectivity of oxybutynin in the same model.

Kᵢ values for compounds tested were found to range from about 5 nM to about 10 μM for M₂ receptors, and from about 0.5 nM to about 10 μM for M₃ receptors. For example, for M₂ receptors, Kᵢ values can range from about 5 nM to about 1 μM, or from about 5 nM to about 500 nM, or from about 5 nM to about 100 nM, or from about 5 nM to about 20 nM (as compared to about 5 nM for tolteridine). For example, for M₃ receptors, Kᵢ values can range from about 0.5 nM to about 500 nM, or from about 0.5 nM to about 100 nM, or from about 0.5 nM to about 20 nM, or from about 0.5 nM to about 5 nM (as compared to about 4 nM for tolteridine).

Selectivity for bladder pressure inhibition vs. salivation was determined for compound Nos. 24, 55 and 85, and was about 2, similar to that determined for tolteridine.

While the present invention has been described in terms of its specific embodiments, certain modification and equivalents will be apparent to those skilled in the art and are included within the scope of the present invention.

1. A compound Formula 1. 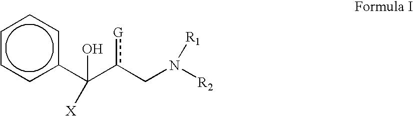
and pharmaceutically acceptable salts, pharmaceutically acceptable solvates, esters, enantiomers, diastereomers, N-oxides, polymorphs, and metabolites thereof, wherein [symbol] represents an optional double bond;
X can be alkyl, alkenyl, alkynyl, cycloalkyl, aryl, heteroaryl, heterocyclyl, heterocyclylalkyl, or heteroarylalkyl.
R₁ can be hydrogen, alkyl, alkenyl, alkynyl, cycloalkyl, alkoxy, aryloxy; —(CH₂)₀₋₂-heterocyclylalkyl, or —(CH₂)₀₋₂-heteroarylalkyl.
R₂ can be —(CH₂)₀₋₂-heteroaryl, —(CH₂)₀₋₂-heterocyclyl, or —(CH₂)₀₋₂-aryl, or R₁ and R₂ may together combine to form a (un)saturated monocyclic or bicyclo ring system containing 0-4 heteroatoms (O, N or S) wherein the ring can be optionally substituted with one or more of alkyl; alkenyl; alkynyl; cycloalkyl; alkaryl; alkoxy; aryloxy; halogen (F, Cl, Br, I); heterocyclyl; heteroaryl; heterocyclylalkyl; heteroarylalkyl; carboxy; nitro; cyano; —C(═O)NRₓR_{y} (wherein Rₓ and R_{y} can be hydrogen, alkyl, alkenyl, alkynyl, cycloalkyl, hydroxy, alkoxy, aryloxy, —(CH₂)₀₋₂-heteroaryl, or —(CH₂)₀₋₂-heterocyclyl); —NRₓR_{y} (wherein Rₓ and R_{y} are as defined above) or —O—C(═O)NRₓR_{y} (wherein Rₓ and R_{y} are as defined above).
G can be —OR(wherein R represents hydrogen or unsubstituted lower (C₁-C₆) alkyl); —NOR (wherein R is the same as defined above); —NHYR′ (wherein R′ is hydrogen, alkyl or aryl and Y is —C(═O), —SO or —SO₂); or oxygen (provided that R₁ and R₂ together does not form a pyrrolidine, 4-hydroxy piperidine, 4-pyrrolidinyl piperidine, piperazine or azabicyclo[3.1.0]Hexane ring).

2. A compound selected from
(R or S) 3-(3-Benzyl-3-azabicyclo[3.1.0]hex-6-ylamino)-1-cyclopentyl-1-phenyl-propane-1,2-diol (Compound No. 1)
(R or S) 3-(3-Benzyl-3-azabicyclo[3.1.0]hex-6-ylamino)-1,1-diphenyl-propane-1,2-diol (Compound No. 2)
3-(3-Azabicyclo[3.1.0]hex-6-ylamino)-1-cyclopentyl-1-phenyl-propane-1,2-diol (Compound No. 3)
(R or S) 3-[(3-Benzyl-3-azabicyclo[3.1.0]hex-6-ylmethyl-amino)]-1-cyclopentyl-1-phenyl-propane-1,2-diol (Compound No. 4)
3-(3-Azabicyclo[3.1.0]hex-6-ylamino)-1,1-diphenyl-propane-1,2-diol (Compound No. 5)
3-(3-Benzyl-3-azabicyclo[3.1.0]hex -6-ylmethyl)-methyl-amino]-1-cyclopentyl-1-phenyl-propane-1,2-diol (Compound No. 6)
(R or S) 3-(3-Benzyl-3-azabicyclo[3.1.0]hex-6-ylamino)-1-cyclopentyl-1-phenyl-propane-1,2-diol (Compound No. 7)
3-[(3-benzyl-3-aza-bicyclo[3.1.0]hex-6-ylmethyl)-methyl-amino]-1-cyclopentyl-1-hydroxy-1-phenyl-propan-2-one-oxime (Compound No. 8)
3-[(3-Azabicyclo[3.1.0]hex-6-ylmethyl)-methyl-amino]-1-cyclopentyl-1-phenyl-propane-1,2-diol (Compound No. 9)
1-Cyclopentyl-1-phenyl-3-(piperazin-1-yl)-propane-1,2-diol (Compound No. 10)
1-Cyclopentyl-3-(4-methyl-piperazin-1-yl)-1-phenyl-propane-1,2-diol (Compound No. 11)
3-(4-Benzyl-piperazin-1-yl)-1-cyclopentyl-1-phenyl-propane-1,2-diol (Compound No. 12);
1-Cyclopentyl-1-phenyl-3-(piperidin-1-yl)-propane-1,2-diol (Compound No. 13)
1-Cyclopentyl-1-hydroxy-1-phenyl-3-(piperidin-1-yl)-propan-2-one (Compound No. 14)
1-Cyclopentyl-1-hydroxy-3-(morpholin-4-yl)-1-phenyl-propane-2-one (Compound No. 15)
1-Cyclopentyl-3-(morpholin-4-yl)-1-phenyl-propane-1,2-diol (Compound No. 16)
3-(1-Benzyl-piperidin-4-ylamino)-1-cyclopentyl-1-hydroxy-1-phenyl-propan-2-one (Compound No. 17)
3-(1-Benzyl-piperidin-4-ylamino)-1-cyclopentyl-1-phenyl-1,2-diol (Compound No. 18)
3-[(1-Benzyl-piperidin-4-yl)-methyl-amino]-1-cyclopentyl-1-hydroxy-1-phenyl-propan-2-one (Compound No. 19)
3-(1-Benzyl-piperidin-4-ylamino)-1-cyclopentyl-1-hydroxy-1-phenyl-propan-2-one oxime (Compound No. 20)
1-Cyclopentyl-3-(3,5-dimethyl-piperazin-1-yl)-1-hydroxy-1-phenyl-propan-2-one (Compound No. 21)
1-Cyclopentyl-3-(3,5-dimethyl-piperazin-1-yl)-1-phenyl-propane-1,2-diol (Compound No. 22)
1-Cyclopentyl-1-hydroxy-3-[methyl-(1-methyl-piperidin-4-yl)-amino]-1-phenyl-propan-2-one (Compound No. 23)
1-Cyclopentyl-3-[methyl-(1-methyl-piperidin-4-yl)-amino]-1-phenyl-propane-1,2-diol (Compound No. 24)
3-[(3-Benzyl-3-aza-bicyclo[3.1.0]hex-6-yl)-methyl-amino]-1-cyclopentyl-1-hydroxy-1-phenyl-propan-2-one (Compound No. 25)
3-[(3-Benzyl-3-aza-bicyclo[3.1.0]hex-6-yl)-methyl-amino]-1-cyclopentyl-1-phenyl-propane-1,2-diol (Compound No. 26)
3-[(3-Aza-bicyclo[3.1.0]hex-6-yl)-methyl-amino]-1-cyclopentyl-1-hydroxy-1-phenyl-propan-2-one (Compound No. 27)
3-(1-Benzyl-piperidin-4-ylamino)-1-cyclopentyl-1-hydroxy-1-phenyl-propan-2-one (Compound No. 28)
3-(1-Benzyl-pyrrolidin-3-ylamino)-1-cyclopentyl-1-propan-1,2-diol (Compound No. 29)
1,1-Diphenyl-3-piperazin-1-yl-propane-1,2-diol (Compound No. 30)
3-(4-Methyl-piperazin-1-yl)-1,1-diphenyl-propane-1,2-diol (Compound No. 31)
1-Hydroxy-3-[methyl-(1-methyl-piperidin-4yl)-amino)-1,1-diphenyl-propan-2-one (Compound No. 32)
3-[Methyl-1-methyl-piperidin-4-yl)-amino]-1,1-diphenyl-propane-1,2-diol (Compound No. 33)
3-(1-Benzyl-pyrrolidin-3-ylamino)-1-cyclopentyl-1-hydroxy-1-phenyl-propane-2-one (Compound No. 34)
3-[(1-Benzyl-pyrrolidin-3-yl)-methyl-amino]-1-cyclopentyl-1-hydroxy-1-phenyl-propan-2-one (Compound No. 35)
3-[4-(4-Chloro-benzyl)-piperazin-1-yl]-1-cyclopentyl-1-phenyl-propane-1,2-diol (Compound No. 36)
3-[4-(2-Benzo[1,3]dioxol-5-yl-ethyl)-piperazin-1-yl]-1-cyclopentyl-1-phenyl-propane-1,2-diol (Compound No. 37)
3-[4-(2-Benzo[1,3]dioxol-5-yl-ethyl)-piperazin-1-yl]-1-cyclopentyl-1-phenyl-propane-1,2-diol (Compound No. 38)
1-Cyclopentyl-1-hydroxy-3-(3-methyl-piperazin-1-yl)-1-phenyl-propan-2-one (Compound No. 39)
1-Cyclopentyl-3-(3-methyl-piperazin-1-yl)-1-phenyl-propane-1,2-diol (Compound No. 40)
1-Cyclopentyl-3-[1,4]diazepan-1-yl-1-hydroxy-1-phenyl-propan-2-one (Compound No. 41)
1-Cyclopentyl-3-[1,4]diazepan-1-yl-1-phenyl-propane-1,2-diol (Compound No. 42)
3-[1,4′]Bipiperidinyl-1′-yl-1-cyclopentyl-1-hydroxy-1-phenyl-propan-2-one (Compound No. 43)
3-[1,4′]Bipiperidinyl-1′-yl-1-cyclopentyl-1-phenyl-propane-1,2-diol (Compound No. 44)
(2R)-1-Cyclopentyl-3-{[2-(dimethylamino)ethyl]amino}-1-hydroxy-1-phenylacetone (Computer No. 45)
1-Cyclopentyl-3-[1′-(3-cyclopentyl-2,3-dihydroxy-3-phenyl-propyl)-[4,4′]bipiperidinyl-1-yl]-1-phenyl-propane-1,2-diol (Compound No. 46)
3-(1-Benzyl-pyrrolidin-3-ylamino)-1,1-diphenyl-propane-1,2-diol (Compound No. 47)
1-Cyclopentyl-3-[4-(3-methyl-but-2-enyl)-piperazin-1-yl]-1-phenyl-propane-1,2-diol (Compound No. 48)
3-[4-(2-Benzo[1,3]dioxol-5-yl-ethyl)-[1,4]diazepan-1-yl]-1-cyclopentyl-1-hydroxy-1-phenyl-propan-2-one (Compound No. 49)
1-Cyclopentyl-3-{4-[2-(2,3-dihydro-benzofuran-5-yl)-ethyl]-[1,4]diazepan-1-yl}-1-hydroxy-1-phenyl-propan-2-one (Compound No. 50)
3-[4-(2-Benzo[1,3]dioxol-5-yl-ethyl)-[1,4]diazepan-1-yl]-1-cyclopentyl-1-phenyl-propane-1,2-diol (Compound No. 51)
1-Cyclopentyl-3-{4-[2-(2,3-dihydro-benzofuran-5-yl)-ethyl]-[1,4]diazepan-1-yl}-1-phenyl-propane-1,2-diol (Compound No. 52)
1-Cyclopentyl-3-dimethylamino-1-hydroxy-1-phenyl-propan-2-one (Compound No. 53)
1-Cyclopentyl-3-dimethylamino-1-phenyl-propane-1,2-diol (Compound No. 54)
3-[1,4]Diazepan-1-yl-1-hydroxy-1,2-diphenyl-propan-2-one (Compound No. 55)
3-[1,4]Diazepan-1-yl-1,1-diphenyl-propane-1,2-diol (Compound No. 56)
3-[4-(2-Benzo[1,3]dioxol-5-yl-ethyl)-[1,4]diazepan-1-yl]-1-hydroxy-1,1-diphenyl-propan-2-one (Compound No. 57)
3-[4-(2-Benzo[1,3]dioxol-5-yl-ethyl)-[1,4]diazepan-1-yl]-1,1-diphenyl-propane-1,2-diol (Compound No. 58)
1-Cyclopentyl-3-(4-hydroxy-piperidin-1-yl)-1-phenyl-propane-1,2-diol (Compound No. 59)
1-Cyclopentyl-3-(2-dimethylamino-ethylamino)-1-phenyl-propane-1,2-diol (Compound No. 60)
1-Cyclopentyl-1-hydroxy-3-(4-methyl-[1,4]diazepan-1-yl)-1-phenyl-propan-2-one (Compound No. 61)
1-Cyclopentyl-3-(4-methyl-[1,4]diazepan-1-yl)-1-phenyl-propane-1,2-diol (Compound No. 62)
1-[1,4]Diazepan-1-yl-3-hydroxy-4-methyl-3-phenyl-pentan-2-one (Compound No. 63)
1-[1,4]Diazepan-1-yl-4-methyl-3-phenyl-pentane-2,3-diol (Compound No. 64)
1-Cyclopentyl-1-hydroxy-3-imidazol-1-yl-1-phenyl-propan-2-one (Compound No. 65)
1-Cyclopentyl-3-imidazol-1-yl-1-phenyl-propane-1,2-diol (Compound No. 66)
1-Cyclopentyl-1-hydroxy-3-(2-methyl-imidazol-1-yl)-1-phenyl-propan-2-one (Compound No. 67)
1-Cyclopentyl-3-(2-methyl-imidazol-1-yl)-1-phenyl-propane-1,2-diol (Compound No. 68)
1-Cyclopentyl-1-hydroxy-3-(2-isopropyl-imidazol-1-yl)-1-phenyl-propan-2-one (Compound No. 69)
1-Cyclopentyl-3-(2-isopropyl-imidazol-1-yl)-1-phenyl-propane-1,2-diol (Compound No. 70)
1-Cyclopentyl-1-hydroxy-3-(2-methyl-4,5-dihydro-imidazol-1-yl)-1-phenyl-propan-2-one (Compound No. 71)
1-Cyclopentyl-1-hydroxy-1-phenyl-3-pyrrolidin-1-yl-propan-2-one (Compound No. 72)
1-Cyclopentyl-1-phenyl-3-pyrrolidin-1-yl-propane-1,2-diol (Compound No. 73)
3-Azepan-1-yl-1-cyclopentyl-1-hydroxy-1-phenyl-propan-2-one (Compound No. 74)
3-Azepan-1-yl-1-cyclopentyl-1-phenyl-propane-1,2-diol (Compound No. 75)
1-Cyclopentyl-1-hydroxy-3-(3-hydroxy-piperidin-1-yl)-1-phenyl-propan-2-one (Compound No. 76)
1-Cyclopentyl-3-(3-hydroxy-piperidin-1-yl)-1-phenyl-propane-1,2-diol (Compound No. 77)
3-(4-Benzyl-piperidin-1-yl)-1-cyclopentyl-1-hydroxy-1-phenyl-propan-2-one (Compound No. 78)
3-[4-(2-Benzo[1,3]dioxol-5-yl-ethyl)-[1,4]diazepan-1-yl]-1-cyclopentyl-1-phenyl-propane-1,2-diol (Compound No. 79)
1-Cyclopentyl-3-(2-dimethylamino-ethylamino)-1-phenyl-propane-1,2-diol (Compound No. 80)
(2R)-1-Cyclopentyl-1-hydroxy-3-(2-methyl-imidazol-1-phenyl-propan-2-one (Compound No. 81)
5-(3-Cyclopentyl-3-hydroxy-2-oxo-3-phenyl-propyl)-2,5-diaza-bicyclo[2.2.1]heptane-2-carboxylic acid tert-butyl ester (Compound No. 82)
Methanesulfonic acid 3-(3-cyclopentyl-3-hydroxy-2-oxo-3-phenyl-propyl)-3-aza-bicyclo[3.2.1]oct-8-yl ester (Compound No. 83)
Methanesulfonic acid 3-(3-cyclopentyl-3-hydroxy-2-oxo-3-phenyl-propyl)-3-aza-bicyclo[3.2.1]oct-8-yl ester (Compound No. 84)
(2R)-1-Cyclopentyl-1-hydroxy-3-(2-methyl-4,5-dihydro-imidazol-1-yl)-1-phenyl-propan-2-one (Compound No. 85)

3. A compound of claim 1, wherein
G is OH, NOH, or oxygen;
X is aryl or cycloalkyl;
R₁ is H or alkyl;
R₂ is optionally substituted heterocyclic, or optionally substituted alkylhetorocyclic, or
R₁ and R₂ join together to form optionally substituted heterocyclic, or optionally substituted alkylhetorocyclic.

4. A pharmaceutical composition comprising a compound of claim 1, and a pharmaceutically acceptable carrier.

5. A method of treatment or prophylaxis of an animal or a human suffering from a disease or disorder mediated through muscarinic receptors, the method comprising administration of a therapeutically effective amount of a compound of claim 1.

6. A method of treatment or prophylaxis of an animal or a human suffering from a disease or disorder mediated through muscarinic receptors, the method comprising administration of a therapeutically effective amount of a composition of claim 4.

7. The method of claim 5, wherein the disease or disorder is selected from bronchial asthma, chronic obstructive pulmonary disorders (COPD), pulmonary fibrosis, and the like; urinary system which induce such urine disorders as urinary incontinence, lower urinary tract symptoms (LUTS), etc.; and gastrointestinal system such as irritable bowel syndrome, obesity, diabetes and gastrointestinal hyperkinesis.

8. (canceled)

9. (canceled)

10. (canceled)

11. (canceled)

12. (canceled)

13. (canceled)

14. (canceled)

15. (canceled)

16. (canceled)

17. (canceled)
